SEQUENCE LISTING (34 sequences)

SEQ ID NO: 1 - DNA, 297 bp, Human papillomavirus
  CDS (1)..(297)
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagctt

SEQ ID NO: 2 - protein, 99 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKL

SEQ ID NO: 3 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 4 - protein, 98 aa, Human papillomavirus
MHGDTPTLHEYMLDLQPETTDLYGYGQLNDSSEEEDEIDGPAGQAEPDRAHYNIVTFCCKCDSTLRLCVQSTHVDIRTLEDLLMGTLGIVCPICSQKP

SEQ ID NO: 5 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 6 - protein, 151 aa, Human papillomavirus
MFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 7 - protein, 151 aa, Human papillomavirus
MFQDPQERPRKLPQLCTELQTTIHDIILECVYCKQQLLRREVYDFAFRDLCIVYRDGNPYAVGDKCLKFYSKISEYRHYCYSLYGTTLEQQYNKPLCDLLIRCINGQKPLCPEEKQRHLDKKQRFHNTRGRWTGRCMSCCRSSRTRRETQL

SEQ ID NO: 8 - DNA, 1899 bp, Homo sapiens
gtccgtactgcagagccgctgccggagggtcgttttaaagggccgcgttgccgccccctcggcccgccatgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagcttgccgcgccaaataatgtctctgtgagactcgagaactttcatttttttccaggctggttcggatttggggtggattttggttttgttcccctcctccactctcccccaccccctccccgccctttttttttttttttttaaactggtattttatcctttgattctccttcagccctcacccctggttctcatctttcttgatcaacatcttttcttgcctctgtgccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctc

SEQ ID NO: 9 - DNA, 1254 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtag

SEQ ID NO: 10 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 11 - protein, 418 aa, Lepus americanus
MLLPVPLLLGLLGLAAAEPVVYFKEQFLDGDGWTERWIESKHKSDFGKFVLSSGKFYGDQEKDKGLQTSQDARFYALSARFEPFSNKGQPLVVQFTVKHEQNIDCGGGYVKLFPAGLDQKDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDANIYAYDSFAVLGLDLWQVKSGTIFDNFLITNDEAYAEEFGNETWGVTKTAEKQMKDKQDEEQRLKEEEEEKKRKEEEEAEEDEEDKDDKEDEDEDEEDKDEEEEEAAAGQAKDEL

SEQ ID NO: 12 - DNA, 1257 bp, Lepus americanus
atgctgctccctgtgccgctgctgctcggcctgctcggcctggccgccgccgagcccgtcgtctacttcaaggagcagtttctggacggagatgggtggaccgagcgctggatcgaatccaaacacaagtccgattttggcaaattcgtcctcagttcgggcaagttctacggcgatcaggagaaagataaagggctgcagaccagccaggacgcccgcttctacgccctgtcggcccgattcgagccgttcagcaacaagggccagccactggtggtgcagttcaccgtgaaacacgagcagaacattgactgcgggggcggctacgtgaagctgtttccggccggcctggaccagaaggacatgcacggggactctgagtacaacatcatgtttggtcctgacatctgtggccccggcaccaagaaggttcacgtcatcttcaactacaagggcaagaacgtgctgatcaacaaggacatccgttgcaaggacgacgagttcacacacctgtacacgctgatcgtgcggccggacaacacgtatgaggtgaagattgacaacagccaggtggagtcgggctccctggaggatgactgggacttcctaccccccaagaagataaaggacccagatgcctcgaagcctgaagactgggacgagcgggccaagatcgacgaccccacggactccaagcccgaggactgggacaagcccgagcacatccccgacccggacgcgaagaagcccgaagactgggacgaagaaatggacggagagtgggagccgccggtgattcagaaccccgagtacaagggtgagtggaagccgcggcagatcgacaaccccgattacaaaggcacctggatccaccccgaaatcgacaaccccgagtactcgcccgacgctaacatctatgcctacgacagctttgccgtgctgggcttggacctctggcaggtcaagtcgggcaccatcttcgacaacttcctcatcaccaacgatgaggcgtacgcagaggagtttggcaacgagacgtggggcgtcaccaagacggccgagaagcagatgaaagacaagcaggacgaggagcagcggctgaaggaggaggaggaggagaagaagcggaaggaggaggaggaggccgaggaggacgaggaggacaaggacgacaaggaggacgaggatgaggacgaggaggacaaggacgaggaggaggaggaggcggccgccggccaggccaaggacgagctgtag

SEQ ID NO: 13 - protein, 416 aa, Rattus sp.
MLLSVPLLLGLLGLAAADPAIYFKEQFLDGDAWTNRWVESKHKSDFGKFVLSSGKFYGDQEKDKGLQTSQDARFYALSARFEPFSNKGQTLVVQFTVKHEQNIDCGGGYVKLFPGGLDQKDMHGDSEYNIMFGPDICGPGTKKVHVIFNYKGKNVLINKDIRCKDDEFTHLYTLIVRPDNTYEVKIDNSQVESGSLEDDWDFLPPKKIKDPDAAKPEDWDERAKIDDPTDSKPEDWDKPEHIPDPDAKKPEDWDEEMDGEWEPPVIQNPEYKGEWKPRQIDNPDYKGTWIHPEIDNPEYSPDANIYAYDSFAVLGLDLWQVKSGTIFDNFLITNDEAYAEEFGNETWGVTKAAEKQMKDKQDEEQRLKEEEEDKKRKEEEEAEDKEDEDDRDEDEDEEDEKEEDEEDATGQAKDEL

SEQ ID NO: 14 - protein, 170 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTH

SEQ ID NO: 15 - protein, 109 aa, Homo sapiens
LYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQ

SEQ ID NO: 16 - protein, 138 aa, Homo sapiens
IDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 17 - DNA, 540 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaac

SEQ ID NO: 18 - DNA, 267 bp, Homo sapiens
acctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccct

SEQ ID NO: 19 - DNA, 444 bp, Homo sapiens
gagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctg

SEQ ID NO: 20 - DNA, 5970 bp, Artificial Sequence
  Description of Artificial Sequence Synthetic plasmid nGVL4a-CRT/E7
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 21 - DNA, 37 bp, Artificial sequence
  Description of Artificial Sequence Synthetic primer
ctgatctctacggttatgggcaattaaatgacagctc

SEQ ID NO: 22 - DNA, 36 bp, Artificial sequence
  Description of Artificial Sequence Synthetic primer
gagctgtcatttaattgcccataaccgtagagatca

SEQ ID NO: 23 - DNA, 31 bp, Artificial sequence
  Description of Artificial Sequence Synthetic primer
aaagtcgacatgctgctatccgtgccgctgc

SEQ ID NO: 24 - DNA, 26 bp, Artificial sequence
  Description of Artificial Sequence Synthetic primer
gaattcgttgtctggccgcacaatca

SEQ ID NO: 25 - DNA, 30 bp, Artificial sequence
  Description of Artificial Sequence Synthetic primer
ccggtctagaacgctgctccctgtgccgct

SEQ ID NO: 26 - DNA, 30 bp, Artificial sequence
  Description of Artificial Sequence Synthetic primer
cccgaattcgttgtccgggccgcacgatca

SEQ ID NO: 27 - DNA, 32 bp, Artificial sequence
  Description of Artificial Sequence Synthetic primer
tgctctagaatgtacaagggtgagtggaagcc

SEQ ID NO: 28 - DNA, 30 bp, Artificial sequence
  Description of Artificial Sequence Synthetic primer
ccggaattccagctcgtccttggcctggcc

SEQ ID NO: 29 - DNA, 31 bp, Artificial sequence
  Description of Artificial Sequence Synthetic primer
tgctctagaatgtacaagggtgagtggaagc

SEQ ID NO: 30 - DNA, 29 bp, Artificial sequence
  Description of Artificial Sequence Synthetic primer
ccggaattccagctcgtccttggcctggc

SEQ ID NO: 31 - DNA, 24 bp, Artificial sequence
  Description of Artificial Sequence Synthetic primer
ggggaattcatggagatacaccta

SEQ ID NO: 32 - DNA, 23 bp, Artificial sequence
  Description of Artificial Sequence Synthetic primer
ggtggatccttgagaacagatgg

SEQ ID NO: 33 - protein, 9 aa, Human papillomavirus
RAHYNIVTF

SEQ ID NO: 34 - protein, 4 aa, Artificial Sequence
  Description of Artificial Sequence Synthetic peptide
KDEL

(19) United States
(12) Patent Application Publication
(10) Pub. No.: US 2008/0102084 A1
(43) Pub. Date: May 1, 2008

(54) Anti-cancer DNA Vaccine Employing Plasmids Encoding Mutant Oncoprotein Antigen and Calreticulin

(76) Inventors: Tzyy-Choou Wu, Stevenson, MD (US); Chien-Fu Hung, Timonium, MD (US)

Correspondence Address: Foley Hoag, LLP (w/JHV), World Trade Center West, 155 Seaport Blvd, Boston, MA, 02210-2600, US

(21) Appl. No.: 11/828,807

(22) Filed: Jul. 26, 2007

Related U.S. Application Data
(63) Continuation-in-part of application No. PCT/US06/02707, filed on Jan. 26, 2006.
(60) Provisional application No. 60/647,150, filed on Jan. 26, 2005.
(60) Provisional application No. 60/647,341, filed on Jan. 26, 2005.

(51) Int. Cl. A61K 31/70; A61K 39/00; A61P 43/00; C07K 14/00; C12N 15/00

(52) U.S. Cl. 424/204.1; 435/320.1; 514/44; 530/350

(57) ABSTRACT

Novel nucleic acid vectors comprising sequences encoding (a) calreticulin or a domain thereof, and (b) an antigen, such as human papillomavirus oncoproteins E7 or E6 in detoxified form, are disclosed, as are methods for using such vectors to induce antigen-specific immune responses and to treat or prevent development of tumors.

### CROSS-REFERENCE TO RELATED APPLICATIONS

This application is a continuation-in-part of PCT/US2006/002707 filed on Jan. 26, 2006, which claims priority to U.S. Provisional Application No. 60/647,150 filed on Jan. 26, 2005, and U.S. Provisional Application No. 60/647,341 filed on Jan. 26, 2005. The contents of each of these applications are hereby incorporated by reference in their entirety.

### BACKGROUND OF THE INVENTION

1. Field of the Invention

The present invention in the fields of molecular biology, immunology and medicine relates to chimeric nucleic acid molecules that encode an antigen, optionally a signal peptide, and an immunogenicity-potentiating polypeptide (“IPP”) such as calreticulin (CRT), and their uses a immunogenic compositions to induce and enhance immune responses, primarily cytotoxic T lymphocyte responses to specific antigens such as tumor or viral antigens.

2. Description of the Background Art

Cytotoxic T lymphocytes (CTL) are critical effectors of anti-viral and antitumor responses (reviewed in Chen, C H et al., J Biomed Sci. 5: 231-52, 1998; Pardoll, D M. Nature Med 4:525-31, 1998; Wang, R F et al., Immunol Rev. 170:85-100, 1999). Activated CTL are effector cells that mediate antitumor immunity by direct lysis of their target tumor cells or virus-infected cells and by releasing of cytokines that orchestrate immune and inflammatory responses that interfere with tumor growth or metastasis, or viral spread. Depletion of CD8⁺ CTL leads to the loss of antitumor effects of several cancer vaccines (Lin, K-Y et al., Canc Res. 56:21-6, 1996; Chen, C-H et al., Canc Res. 60: 035-42, 2000). Therefore, the enhancement of antigen presentation through the MHC class I pathway to CD8⁺ T cells has been a primary focus of cancer immunotherapy.

DNA vaccines have emerged as an attractive approach for antigen-specific cancer immunotherapy. DNA vaccines offer many advantages over more conventional vaccines, such as peptide or attenuated live pathogens. One advantage is that DNA vaccines are reasonably stable and can be easily prepared and harvested in large quantities. Additionally, naked plasmid DNA is relatively safe and can be repeatedly administered without adverse effects. Furthermore, because DNA is able to be maintained in cells for long-term expression of the encoded antigen, maintenance of immunologic memory is possible (see, Donnelly, J J et al., Annu Rev Immunol 1997, 15:617-648; Pardoll, supra; Robinson, H L, Vaccine 15:85-787, 1997; Gurunathan, S et al., Annu Rev Immunol 18:927-74, 2000).

However, one limitation of these vaccines is their lack of potency, since the DNA vaccine vectors generally do not have the intrinsic ability to be amplified and to spread in vivo as do some replicating viral vaccine vectors. Furthermore, some tumor antigens such as the E7 protein of human papillomavirus-16 (“HPV-16”) are weak immunogens (Chen et al., 2000, supra). Therefore, there is a need in the art for strategies to enhance DNA vaccine potency, particularly for more effective cancer and viral immunotherapy.

One strategy taken by the present inventors in the present invention to enhance the presentation of antigen through the MHC class I pathway to CD8⁺ T cells is the exploitation of the features of certain polypeptides to target antigenic polypeptide to which they are fused. Such polypeptide are referred to collectively herein as “immunogenicity-potentiating (or -promoting) polypeptide” or “IPP” to reflect this general property, even though these IPP's may act by any of a number of cellular and molecular mechanisms that may or may not share common steps. This designation is intended to be interchangeable with the term “targeting polypeptide.” Inclusion of nucleic acid sequences that encode polypeptides that modify the way the antigen encoded by molecular vaccine is “received” or “handled” by the immune system serve as a basis for enhancing vaccine potency.

Calreticulin

Calreticulin (CRT), an abundant 46 kilodalton (kDa) protein located in the lumen of the cell's endoplasmic reticulum (ER), displays lectin activity and participates in the folding and assembly of nascent glycoproteins. See, e.g., Nash., Mol Cell Biochem 135:71-8, 1994; Hebert, J Cell Biol 139:613-23, 1997; Vassilakos, A, Biochemistry 37:3480-90, 1998; Spiro, J Biol Chem 271:11588-94, 1996). Rabbit, human, mouse, and rat CRT's show >90% sequence identity (Michalak, M et al., Biochem J, 344 Pt 2:281-92, 1999).

CRT associates with peptides transported into the ER by transporters that are associated with antigen processing, such as TAP-1 and TAP-2 (Spee, Eur J Immunol 27:2441-9, 1997). CRT also forms complexes with peptides in vitro. Upon administration to mice, these complexes, elicited peptide-specific CD8+ T cell responses (Basu, J Exp Med 189:797-802, 1999; Nair, J Immunol 162:6426-32, 1999). CRT purified from murine tumors elicited immunity specific for the tumor from which the CRT was taken, but not for an antigenically distinct tumor (Basu, supra). By pulsing mouse dendritic cells (DCs) in vitro with a CRT-peptide complex, the peptide was re-presented by MHC class I molecules on the DCs to stimulate a peptide-specific CTL response (Nair, supra).

The CRT protein is composed of three domains, the N-domain, P-domain and C-domain. The N-domain (residues 1-180), also known as vasostatin, is highly conserved among CRTs from different species (Krause, K H et al., Cell 88:439-43, 1997). The N-domain interacts with the DNA-binding domain of the glucocorticoid receptor in vitro, with rubella virus RNA (Singh, N K et al., Proc Natl Acad Sci USA 91:12770-74, 1994), with α-integrin (Rojiani, M V et al., Biochemistry 30:9859-66, 1991), and with protein disulphide-isomerase (PDI) and ER protein 57 (ERp57) (Corbett, E F et al., J Biol Chem 1999, 274:6203-11, 1999). The N-domain of calreticulin also inhibits proliferation of endothelial cells and suppresses angiogenesis (Pike, S E et al. J Exp Med 188:2349-56, 1998). The P-domain (residues 181-280) is rich in Pro and contains two sets of three sequence repeats. This domain binds Ca⁺⁺ with high affinity (Baksh, S et al., J Biol Chem 266:21458-65, 1991). The P-domain is thought to be critical for the lectin-like chaperone activity of CRT (Vassilakos et al., supra) and also interacts with PDI and perforin (Andrin, C et al., Biochemistry 37:10386-94, 1998; Fraser, S A et al., Biochem Cell Biol 76:881-7, 1998). The C-terminal region of CRT is highly acidic and terminates with the KDEL ER retrieval sequence Michalak, M et al., J Biol Chem 271:29436-45, 1996). The C domain of CRT binds to Ca (Baksh et al., supra) and to blood-clotting factors (Kuwabara, K et al., J Biol Chem 270:8179-87, 1995) and inhibits injury-induced restenosis (Dai, E et al., Arterioscler Thromb Vasc Biol 17:2359-68, 1997).

CRT also has anti-angiogenic effects. CRT and its N domain are endothelial cell inhibitors that can suppress tumor growth (Pike, S E, Blood 94:2461-8, 1999). Tumor growth and metastasis depend on the existence of an adequate blood supply. As tumors grow larger, adequate blood supply is often ensured by new vessel formation, a process termed angiogenesis. Therapeutic agents that target and damage tumor vasculature can prevent or delay tumor growth and even promote regression or dormancy.

Immunogenic Constructs with HPV E7 as a Model Antigen

The present inventors and their colleagues previously developed several intracellular targeting and intercellular spreading strategies to enhance DNA vaccine potency using various immunogenicity-potentiating polypeptide (“IPP”). See for example, publications of the present inventors and their colleagues: Hung, C F et al., J Virol 76:2676-82, 2002; Cheng, W F et al., J Clin Invest 108:669-78, 2001; Hung, C F et al., J Immunol 166:5733-40, 2001; Chen, C H et al., Gene Ther 6:1972-81, 1999; Ji, H et al., Hum Gene Ther 10:2727-40, 1999; Chen, C H et al., Cancer Res 60:1035-42, 2000; U.S. Pat. No. 6,734,173, WO 01/29233; WO03/085085; WO 02/012281; WO 02/061113).

Among these strategies was the linkage of linking antigen to the intracellular targeting moiety CRT. The present inventors and their colleagues were the first to provide naked DNA and self-replicating RNA vaccines that incorporated CRT (or other IPPs). The present inventors and their colleagues also demonstrated that linking antigen to Mycobacterium tuberculosis heat shock protein 70 (HSP70) or its C-terminal domain, domain II of Pseudomonas aeruginosa exotoxin A (ETA(dII)), or the sorting signal of the lysosome-associated membrane protein type 1 (Sig/LAMP-1) enhanced DNA vaccine potency compared to compositions comprising only DNA encoding the antigen of interest. To enhance MHC class II antigen processing, the present inventors' colleagues (Lin, K Y et al., Cancer Res 56: 21-6, 1996) linked the sorting signals of the lysosome-associated membrane protein (LAMP-1) to the cytoplasmic/nuclear human papilloma virus (HPV-16) E7 antigen, creating a chimera (Sig/E7/LAMP-1). These findings point to the importance of adding an “element” to an antigenic composition at the DNA level to enhance in vivo potency of a recombinant DNA vaccine.

Intradermal administration of DNA vaccines via gene gun in vivo have proven to be an effective means to deliver such vaccines into professional antigen-presenting cells (APCs), primarily dendritic cells (DCs), which function in the uptake, processing, and presentation of antigen to T cells. The interaction between APCs and T cells is crucial for developing a potent specific immune response.

Various DNA constructs described by the present inventors or others in the prior art, have resulted in certain combinations that induced a heightened immune response in experimental animals. However, none of these vaccines have been ideally designed for use in humans where administration may be limited for practical or other reasons to intramuscular (i.m.) injection. Because direct transduction of professional APCs in muscle tissue is not likely to occur due to paucity of such cells in muscle. That leaves cross-priming as the most likely mechanism for the induction of heightened immunity in humans. Optimizing vaccine constructs for cross priming requires that an element be added that promotes the secretion of the expressed polypeptide antigenic moiety, preferably as a fusion polypeptide with a molecule that promotes antigen processing via the MHC class I pathway. Moreover, it best to used plasmid constructs that are know to be safe and effective in humans. Finally, in the case of HPV oncoprotein antigens, it is also important to “detoxify” the protein that is to be expressed so that it will not act as an oncogenic transforming agent. It is to such constructs with the aforementioned advantageous properties that the present invention is directed.

### SUMMARY OF THE INVENTION

The present inventors have designed and disclose herein an immunotherapeutic strategy that combines antigen-encoding DNA vaccine compositions in which the antigen-encoding DNA is linked to human CRT or a homologue thereof, a domain or fragment thereof, or other functional derivative that promotes processing of the antigen via the MHC class I pathway and enhanced immunogenicity.

The present invention is directed to a nucleic acid molecule that is an expression vector expressable in a eukaryotic cell, and encodes a chimeric or fusion polypeptide, which molecule comprises:
(a) a first nucleic acid sequence, preferably SEQ ID NO:9, encoding a first polypeptide which is calreticulin (CRT), preferably SEQ ID NO:10, or a biologically active homologue, domain or fragment thereof,
   which homologue, domain or fragment (i) forms complexes with peptides in vitro; (ii) when expressed in a cell, participates in folding and assembly of nascent glycoproteins, (iii) when expressed in a cell, associates with peptides transported into the endoplasmic reticulum by transporters that are associated with antigen processing, and/or (iv) inhibits angiogenesis;
(b) a second nucleic acid sequence that is linked in frame to the first nucleic acid sequence and that encodes an antigenic polypeptide or peptide that preferably comprises an epitope that binds to a MHC class I protein, and
(c) operably linked thereto, a promoter active in the eukaryotic cell and, optionally, one or more regulatory elements that enhance expression of the nucleic acid in the cell.

The first nucleic acid sequence may encode one or more CRT fragments or domain selected from the group consisting of (a) N-CRT, (b) P-CRT, (c) S-CRT and (d) a biologically active variant of (a), (b) or (c) 5. Preferably, N-CRT is SEQ ID NO:14), P-CRT is SEQ ID NO:15, S-CRT is SEQ ID NO:16) or each of the domains is a homologue of N-CRT, P-CRT or S-CRT. In a preferred embodiment of the above nucleic acid molecule that encodes a CRT domain, the first nucleic acid sequence is SEQ ID NO:17 or encodes N-CRT (SEQ ID NO:14). The first nucleic acid sequence may encodes any two or more of N-CRT (SEQ ID NO:14), P-CRT (SEQ ID NO:15), C-CRT (SEQ ID NO:16) or any combination thereof.

In the above nucleic acid molecule, the antigen is preferably one which is present on, or cross-reactive with an epitope of, a pathogenic organism, cell, or virus, most preferably the virus is a human papilloma virus such as HPV-16 and the antigen is (a) an E7 polypeptide of HPV having the sequence SEQ ID NO:3; (b) an E6 polypeptide of HPV having the sequence SEQ ID NO:6; (c) an in-frame linked combination of E6 and E7 in either orientation; (d) an antigenic fragment of E6 or E7; (e) a non-oncogenic mutant or variant of E6 or E7; or (f) an in-frame linked non-oncogenic mutant/variant E7-E6 combination in either orientation. In a preferred embodiment of the nucleic acid molecule, the antigen is a non-oncogenic mutant or variant of the E7 polypeptide.

A preferred mutant/variant of the E7 polypeptide differs from SEQ ID NO:3 by one or more of the following substitutions: (a) Cys at position 24 to Gly or Ala; (b) Glu at position 26 to Gly or Ala; and (c) Cys at position 91 to Gly or Ala, most preferably a polypeptide with the sequence SEQ ID NO:4.

A preferred mutant E6 polypeptide has the sequence SEQ ID NO:6. A preferred non-oncogenic mutant/variant of the E6 differs from SEQ ID NO:6 by one or more of the following substitutions: (a) Cys at position 63 to Gly or Ala; (b) Cys at position 106 to Gly or Ala; and (c) Ile at position 128 to Thr, most preferably a polypeptide with the sequence SEQ ID NO:7.

The nucleic acid molecule as described above is preferably part of a plasmid, most preferably of the plasmid pNGV4a. In a most preferred embodiment, the nucleic acid is characterized as the plasmid pNGVL4a/CRT/E7(detox), and has the sequence SEQ ID NO:20.

Also provided is a pharmaceutical composition capable of inducing or enhancing an antigen-specific immune response, comprising:
(a) pharmaceutically and immunologically acceptable excipient in combination with;
(b) a composition comprising the above nucleic acid molecule.

The invention is also directed to a method of inducing or enhancing an antigen specific immune response in a subject comprising administering to the subject an effective amount of the above pharmaceutical composition, thereby inducing or enhancing the response. The immune response is preferably one that is mediated at least in part by CD8⁺ cytotoxic T lymphocytes (CTL).

Also provided is a method of inhibiting growth or preventing re-growth of a tumor expressing HPV E7 or E6 protein in a subject, comprising administering to the subject an effective amount of the above pharmaceutical composition, wherein the second nucleic acid sequence encodes one or more epitopes of E7 or E6, respectively, thereby inhibiting the growth or preventing the re-growth.

In all of the above methods, the administering may be by intramuscular injection, by gene gun intradermal administration, or by needle-free jet injection.

In all of the above methods, the subject is preferably a human and the administering is by a intramuscular injection.

Also provided herein is (1) use of the above nucleic acid molecule, for the manufacture of a medicament for inducing or enhancing an antigen specific immune response in a subject, and (2) use of the above nucleic acid molecule for the manufacture of a medicament for inhibiting growth or preventing re-growth of a tumor expressing HPV E7 or E6 protein in a subject.

### BRIEF DESCRIPTION OF THE DRAWINGS

FIGS. 1 & 2. Flow cytometry analysis of IFN-γ-secreting E7-specific CD8+ T cell precursors in mice vaccinated with various recombinant DNA vaccines. Mice (4/group) were immunized with pcDNA3-CRT/E7, pcDNA3-E7/HSP70, pcDNA3-ETA/E7, pcDNA3-Sig/E7/LAMP-1, pcDNA3-E7, and pcDNA3 as described in the Examples. Spleen cells from vaccinated mice were harvested 7 days after a booster vaccination, cultured in vitro with MHC class I-restricted E7(aa 49-57) peptide overnight, and stained for both CD8 and intracellular IFN-γ. FIG. 1 shows representative flow cytometry data. FIG. 2 is a bar graph depicting the number of antigen-specific IFN-γ-secreting CD8⁺ T cell precursors/3×10⁵ spleen cells (mean ±SD). These results are from one representative experiment of two performed.

FIG. 3. In vivo tumor treatment experiments to compare the antitumor effect generated by various DNA vaccine constructs in mice. Mice (5/group) were challenged with 10⁴ TC-1 tumor cells and immunized with various DNA constructs seven days later. Results are expressed as the mean number of lung nodules; bars, ±SD. These results are from one representative experiment of two performed.

FIGS. 4 & 5. Flow cytometric analysis of IFN-γ-secreting E7-specific CD8+ T cell precursors generated by various DNA vaccine constructs in vaccinated mice eight weeks after initial vaccination. Mice (4/group) were immunized with the DNA constructs as described for FIG. 1. Spleen cells from vaccinated mice were harvested 8 weeks after the initial vaccination, cultured in vitro with MHC class I-restricted E7(aa 49-57) peptide overnight, and stained for both CD8 and intracellular IFN-γ. FIG. 4 shows representative flow cytometry results. FIG. 5 is a graph depicting the number of antigen-specific IFN-γ-secreting CD8⁺ T cell precursors/3×10⁵ spleen cells (mean ±SD). These results are from one representative experiment of two performed.

FIG. 6. Long-term in vivo tumor protection experiments to compare the antitumor effect generated by various DNA vaccine constructs in vaccinated mice eight weeks after initial vaccination. Mice (5/group) were immunized and challenged with 10⁵ cells/mouse TC-1 tumor cells as described in the Examples. Results are expressed as the mean number of lung nodules; bars, ±SD. These results are from one representative experiment of two performed.

FIG. 7-9. MHC class I expression of TC-1 P3 (A15) and in vivo tumor protection experiment using TC-1 P3 (A15) tumor cells. FIG. 7 shows results of flow cytometric analysis characterizing MHC class I expression on TC-1 P0 and the TC-1 P3 (A15) subclone. B16 was used as a negative control (dotted line). TC-1 P0 cells are MHC class I positive (thick line), whereas TC-1 P3 (A15) exhibits down-regulated MHC class I expression (filled region). FIG. 8 shows results of an in vivo tumor protection experiments using TC-1 P3 (A15) tumor cells. Mice (5/group) were vaccinated with 2 μg of pcDNA3-E7, pcDNA3-CRT, pcDNA3-CRT/E7 DNA or pcDNA3 empty plasmid. One week after the last vaccination, mice were challenged with 5×10⁴ TC-1 P3 (A15) tumor cells by subcutaneous injection in the right leg. Mice vaccinated with CRT/E7 DNA provided 100% protection against TC-1 P3 (A15) when compared to mice vaccinated with other DNA vaccines (one-way ANOVA, p<0.01). FIG. 9 shows results of in vivo tumor protection experiments using IFN-γ KO mice. Wild type C57BL/6 mice and IFNγ-depleted C57BL/6 mice (5/group) were vaccinated with 2 μg of pcDNA3-CRT/E7 DNA. One week after the last vaccination, mice were challenged with 5×10⁴ TC-1 P3 (A15) tumor cells by subcutaneous injection in the right leg as described above. 100% of wt C57BL/6 mice and only 20% of IFNγ-depleted C57BL/6 mice remained free of tumors.

FIGS. 10 & 11. Intracellular cytokine staining with flow cytometry to demonstrate the number of E7-specific CD8+ T cell precursors in mice vaccinated with pNGVL4a-CRT/E7(detox). One group of C57BL/6 mice was immunized intramuscularly (i.m.) with 50 μg of DNA vaccine and received a booster with the same regimen one week later. Another group was immunized intradermally (i.d.) via gene gun with 2 μg of DNA vaccine and received a booster with the same regimen one week later. Spleen cells were collected one week after the last vaccination. The number of E7-specific IFN-γ-secreting CD8⁺ T cell precursors was analyzed using ICCS followed by flow cytometry. FIG. 10 shows representative flow cytometry results (one representative experiment of two performed). FIG. 11 shows the results in the form of a bar graph showing the number of interferon-secreting T cells/3×10⁵ spleen cells.

FIG. 12 is a schematic diagram of the pNGVL4a/CRT/E7(detox) plasmid vector used for anti-tumor vaccination. Indicated are various cloning sites, promoters and coding sequences.

### DESCRIPTION OF THE PREFERRED EMBODIMENTS

### Partial List of Abbreviations Used

APC, antigen presenting cell; CRT, calreticulin; CTL, cytotoxic T lymphocyte; DC, dendritic cell; ECD, extracellular domain; E6, HPV oncoprotein E6; E7, HPV oncoproteinE7; ELISA, enzyme-linked immunosorbent assay; HPV, human papillomavirus; HSP, heat shock protein; Hsp70, mycobacterial heat shock protein 70; IFNγ, interferon-γ; i.m., intramuscular(ly); i.v., intravenous(ly); MHC, major histocompatibility complex; PBS, phosphate-buffered saline; PCR, polymerase chain reaction; β-gal, β-galactosidase

The present inventors and their colleagues have shown that the linkage of CRT and homologues thereof to human papillomavirus type 16 (HPV-16) oncoprotein E7 dramatically increased E7-specific CD8+ T cell precursors and enhance anti-tumor effects against an E7-expressing tumor in vaccinated mice. This is shown in the Examples below, as well as in other publications by the inventors and their colleagues (e.g., Wu & Hung, WO00212281A1 (pub'd 14-FEB-2002); Peng S et al., J Virol. 2004 78:8468-76, 2004; Kim J W et al., Gene Ther. 11:1011-8, 2004; Peng S et al., Gene Ther. 2005, Sep. 22; Epub; Cheng W F et al., Vaccine 23:3864-74, 2005).

They have now adopted this strategy for clinical trials in patients with HPV-16 associated high-grade squamous intraepithelial lesion of the cervix and in patients with advanced HPV-associated head and neck squamous cell carcinoma. To do so, a CRT/E7 DNA vaccine of a grade consistent with “good manufacturing practice (GMP) requirements was produced in the form of a naked DNA preparation based on the pNGVL4a plasmid developed by the National Gene Vector Lab that has been approved for use in humans. The DNA molecule of the present invention has been termed “pNGVL4a/CRT/E7(detox))”. A similar DNA vaccine for the E6 protein is also described below and is referred to as “pNGVL4a/CRT/E6(detox)”.

The pNGVL4a/CRT/E7(detox) DNA vaccine (see below) was generated using the pNGVL4a vector as a backbone. This vector was originally derived from the pNGVL3 vector, which has been approved for human vaccine trials. The pNGVL4a vector includes two immunostimulatory sequences (tandem repeats of CpG dinucleotides) in the noncoding region. Whereas any other plasmid DNA that can transform either APCs or other cells which, via cross-priming, transfer the antigenic moiety to APCs, is useful in the present invention, pNGVLA4 is preferred because it has already been approved for human therapeutic use.

Intramuscular immunization with a secreted form of an antigen or, as here, a DNA vaccine encoding the antigen together with CRT will generate stronger CTL responses than i.m. immunization with a “cytoplasmic” form of antigen, suggesting that the priming of CTL responses after i.m. DNA immunization is facilitated by the cross-presentation of antigen by non-transfected professional APCs that have acquired the immunogen/antigen indirectly. See, for example, Boyle, J S et al., Int Immunol 1997, 9:1897-1906.

In addition, when an oncoprotein or an epitope thereof is the immunizing moiety, it is preferred to reduce the tumorigenic risk of the vaccine itself. The key target antigen, HPV E7 and E6 are oncogenic. In the preferred embodiments herein the DNA encoding the E7 protein was doubly mutated to a form known as “E7(detox)” by substituting nucleotides resulting in substitution of two amino acids at positions C²⁴G (Cys→Gly) and E²⁶G (Glu→Gly) as described in detail below. These substitutions completely eliminate the capacity of the E7 to binding capacity to Rb, as well as transforming activity. Other substitutions that could achieve the same effect can be readily made by those skilled in the art.

Whereas the present exemplification focused on E7, another embodiment of the present invention comprises an antigenic epitope of the HPV E6 protein, preferably from HPV-16. The E6 proteins from cervical cancer-associated HPV types such as HPV-16 induce proteolysis of the p53 tumor suppressor protein through interaction with E6-AP. Human mammary epithelial cells (MECs) immortalized by E6 display low levels of p53. HPV-16 E6 as well as other cancer-related papillomavirus E6 proteins also binds the cellular protein E6BP (ERC-55). Several different E6 mutations are discussed below after presentation of the “wild type” sequence. Studies describing these mutants (which are incorporated by reference in their entirety) are also discussed in that section.

The present invention also includes the use of a tandem E6-E7 vaccine, using one or more of the mutations described herein to render the oncoproteins inactive with respect to their oncogenic potential in vivo. M C Cassetti et al. (Vaccine 22:520-7, 2004) described Venezuelan equine encephalitis virus replicon particle (VRP) vaccines encoding the HPV16 E6 and E7 genes in which the E6 and E7 genes were fused to create one open reading frame and mutated at four or at five amino acid positions (see below). Thus, the present constructs may include one or more epitopes of E6 and E7, which may be arranged in their native order, resulting in either a E6-E7 or E7-E6 sequence, or shuffled in any way that permits the expressed protein to bear the E6 and E7 antigenic epitope in an immunogenic form and result in immunization of the vaccinated recipient. DNA encoding an amino acid spacers between E6 and E7 or between individual epitopes of these proteins may be introduced into the vector, provided again, that the spacers permit the expression and presentation of the epitopes in an immunogenic manner after they have been expressed by transduced host cells.

The orientation in which the two (or more) component polypeptides of the fusion protein are arranged, and therefore, the order of the encoding nucleic acid fragments in the nucleic acid vector, can in certain cases be altered without affecting immunogenicity of the fusion polypeptides proteins and the efficacy of the DNA vaccine, though one orientation may give better results than another. For example, the HSP70-encoding DNA sequences may be located 5′ or 3′ to the target antigen-encoding sequences, though E7 (or E6)-HSP70 orientation (N- to C-terminal) is preferred. With CRT, as disclosed herein, the CRT-E7 (or E6) N- to C-terminal orientation is preferred. One skilled in the art can routinely test and compare alternative orientations using methods described herein and those conventional in the art, without undue experimentation.

In a preferred embodiment, these IPP-encoding nucleic acid domains are fused in-frame with the antigen DNA so that the chimeric DNA construct encodes a recombinant fusion polypeptide with the antigen N- (or C-) terminal to the IPP. Of course, if a signal peptide, is included, it should be at the N-terminus of a nascent protein.

As has been disclosed in the inventors' patent and other publications and as will be appreciated by those skilled in the art, the present DNA construct encodes a recombinant polypeptide encoding any MHC class I restricted antigen, exemplified herein by mutant (detox) E7.

Sequences of Polypeptides and Nucleic Acids

Plasmid and Vector Sequences

The wild-type HPV E7 sequence (nucleotide sequence is SEQ ID NO: 1 used in the present invention, albeit with several mutations, and the wild-type amino acid sequence is SEQ ID NO:2) is shown below. Underlined codons and amino acids are those which are preferably mutated in the present constructs.

1/1 31/11
atg cat gga gat aca cct aca ttg cat gaa tat atg
tta gat ttg caa cca gag aca act
Met His Gly Asp Thr Pro Thr Leu His Glu Tyr Met
Leu Asp Leu Gln Pro Glu Thr Thr
61/21 91/31
gat ctc tac tgt tat gag caa tta aat gac agc tca
gag gag gag gat gaa ata gat ggt
Asp Leu Tyr Cys Tyr Glu Gln Leu Asn Asp Ser Ser
Glu Glu Glu Asp Glu Ile Asp Gly
121/41 151/51
cca gct gga caa gca gaa ccg gac aga gcc cat tac
aat att gta acc ttt tgt tgc aag
Pro Ala Gly Gln Ala Glu Pro Asp Arg Ala His Tyr
Asn Ile Val Thr Phe Cys Cys Lys
181/61 211/71
tgt gac tct acg ctt cgg ttg tgc gta caa agc aca
cac gta gac att cgt act ttg gaa
Cys Asp Ser Thr Leu Arg Leu Cys Val Gln Ser Thr
His Val Asp Ile Arg Thr Leu Glu
241/81 271/91
gac ctg tta atg ggc aca cta gga att gtg tgc ccc
atc tgt tct cag gat aag ctt
Asp Leu Leu Met Gly Thr Leu Gly Ile Val Cys Pro
Ile Cys Ser Gln Asp Lys Leu

The above sequence differs from the GENEBANK Accession Number NC_{—}001526 for the E7 protein which is:

(SEQ ID NO: 3)

MHGDTPTLHE YMLDLQPETT DLYCYEQLND SSEEEDEIDG
PAGQAEPDRA HYNIVTFCCK CDSTLRLCVQ STHVDIRTLE
DLLMGTLGIV CPICSQKP 97

The HPV 16 E7 protein binds to its target, Rb, through an L-X-C-X-E motif. Mutations at positions Cys24 and Glu26 of this motif destroy Rb binding and degradation. In addition to these two point mutations in E7, a mutation at a third amino acid, Cys91, destroys the single zinc finger in E7. In a preferred embodiment, all wild type amino acids are mutated to Gly. In another embodiment, these residues are mutated to Ala. In fact, they can be mutated to any residue that will permit the protein to be expressed in transduced cells, secreted in immunogenic form, taken up by professional APCs, and presented to T cells in a way that will preserve antigenic specificity, while at the same time preventing or lowering the probability that the protein will have oncogenic transforming capacity. The above statement is true with respect to the HPV E6 protein described below.

Any nucleotide sequence that encodes an E7 polypeptide, or preferably, one of the mutants thereof discussed below, or an antigenic fragment or epitope thereof, can be used in the present vectors.

An antigenic fragment of E7 may include at least or about 30, 50, 75, or 95 amino acids of E7. An antigenic fragment may be at least or about 60%, 70%, 85%, 90%, 95%, 96%, 97%, 98%, or 99% identical to an E7 sequence provided herein. An antigenic fragment may include at least or about 10-25, 25-50, 50-75, or 75-85 amino acids of E7.

A “fragment” of E7 refers to any subset of the molecule, that is, a shorter polypeptide of the full-length protein.

To reduce oncogenic potential of E7 in a construct of this invention, one or more of the following positions of E7 is mutated:

| Original | Mutant | Preferred | Position in |
|---|---|---|---|
| residue | residue | codon mutation | SEQ ID NO: 3 |
| Cys | Gly (or Ala) | TGT→GGT | 24 |
| Glu | Gly (or Ala) | GAG→GGG | 26 |
| | | (or GCG) | |
| Cys | Gly (or Ala) | TGC→GGC | 91 |

The E7 (detox) mutant amino acid sequence (SEQ ID NO:4, below) encoded by the preferred vaccine vector has the mutations shown above—namely—a tgt→ggt mutation resulting in a Cys→Gly substitution at position 24 and a gag→ggg mutation resulting in a Glu→Gly substitution at position 26 of SEQ ID NO:2.

(SEQ ID NO: 4)

MHGDTPTLHE YMLDLQPETT DLYGYGQLND SSEEEDEIDG
PAGQAEPDRA HYNIVTFCCK CDSTLRLCVQ STHVDIRTLE
DLLMGTLGIV CPICSQKP 97

E6 Protein

The wild type HPV E6 amino acid sequence (GENEBANK Accession Number NC_{—}001526) (SEQ ID NO:5) is shown below. This sequence has 158 amino acids.

MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL 158

The preferred amino acid residues to be mutated are underscored above. The studies of E6 mutants discussed below are based upon a shorter E6, the coding sequence of which encodes 151 amino acids, the N-terminal residue of which was counted as Met-8 in SEQ ID NO:5. That shorter version of E6 is shown below as SEQ ID NO:6.

MFQDPQERPR KLPQLCTELQ TTIHDIILEC VYCKQQLLRR
EVYDFAFRDL CIVYRDGNPY AVCDKCLKFY SKISEYRHYC
YSLYGTTLEQ QYNKPLCDLL IRCINCQKPL CPEEKQRHLD
KKQRFHNIRG RWTGRCMSCC RSSRTRRETQ L 151

Again, the preferred amino acid residues to be mutated are underscored.

Any nucleotide sequence that encodes this E6 polypeptide, or preferably, one of the mutants thereof discussed below, or an antigenic fragment or epitope thereof, can be used in the present vectors. Codons may be selected by typical codon usage rules. Other mutations can be tested and used in accordance with the methods described herein, or those described by Cassetti et al., supra.

An antigenic fragment of E6 may include at least or about 30, 50, 75, 95, 125, or 150 amino acids of E6. An antigenic fragment may be at least or about 60%, 70%, 85%, 90%, 95%, 96%, 97%, 98%, or 99% identical to an E6 sequence provided herein. An antigenic fragment may include at least or about 10-25, 25-50, 50-75, 75-85, 85-125, or 125-150 amino acids of E6.

A “fragment” of E6 refers to any subset of the molecule, that is, a shorter polypeptide of the full-length protein.

To reduce oncogenic potential of E6 in a construct of this invention, one or more of the following positions of E6 is mutated:

| | | | |
|---|---|---|---|
| Original | Mutant | Position in | Position in |
| residue | residue | SEQ ID NO: 5 | SEQ ID NO: 6 |

| | | | |
|---|---|---|---|
| Ile | Thr | 135 | 128 |
| Cys | Gly (or Ala) | 70 | 63 |
| Cys | Gly (or Ala) | 113 | 106 |

A form of E6(detox) having these three mutations is shown below as SEQ ID NO:7

MFQDPQERPR KLPQLCTELQ TTIHDIILEC VYCKQQLLRR
EVYDFAFRDL CIVYRDGNPY AVGDKCLKFY SKISEYRHYC
YSLYGTTLEQ QYNKPLCDLL IRCINGQKPL CPEEKQRHLD
KKQRFHNTRG RWTGRCMSCC RSSRTRRETQ L 151

These mutations can be achieved using any appropriate coding sequences by mutation of the coding DNA.

The studies describing these mutants (which are incorporated by reference in their entirety) are discussed below. M. Nguyen et al. (J Virol. 6:13039-48, 2002) described a mutant of HPV-16 E6 deficient in binding α-helix partners which displays reduced oncogenic potential in vivo. This mutant, that involves a replacement of Ile with Thr as position 128 (of SEQ ID NO:6), may be used in accordance with the present invention to make an E6 DNA vaccine that has a lower risk of being oncogenic. This E6(I¹²⁸T) mutant is defective for binding at least a subset of the α-helix partners, including E6AP, the ubiquitin ligase that mediates E6-dependent degradation of the p53 protein,

Cassetti et al., supra, examined the effects of mutations four or five amino acid positions in E6 and E7 to inactivate their oncogenic potential. The following mutations were examined (positions based on SEQ ID NO:3 or 5): E6-C⁶³G; E6 C¹⁰⁶G; E7-C²⁴G, E7-E²⁶G, and E7 C⁹¹G. Vaccines encoding mutant or wild type E6 and E7 proteins elicited comparable CTL responses and generated comparable antitumor responses in several HPV16 E6⁺E7⁺ tumor challenge models: protection from either C3 or TC-1 tumor challenge was observed in 100% of vaccinated mice. Eradication of C3 tumors was observed in approximately 90%. The predicted inactivation of E6 and E7 oncogenic potential was confirmed by demonstrating normal levels of both p53 and Rb proteins in human mammary epithelial cells infected with VRP expressing mutant E6 and E7 genes.

Approaches for Mutagenesis of E6 and E7

The HPV16 E6 protein contains two zinc fingers important for structure and function; one cysteine (C) amino acid position in each pair of C-X-X-C (where X is any amino acid) zinc finger motifs are preferably was mutated at E6 positions 63 and 106 (based on SEQ ID NO:5). Mutants are created, for example, using the Quick Change Site-Directed Mutagenesis Kit (Stratagene, La Jolla, Calif.). HPV16 E6 containing a single point mutation at Cys106 (of Cys 113 per SEQ ID NO:5). Cys neither binds nor facilitates degradation of p53 and is incapable of immortalizing human mammary epithelial cells (MEC), a phenotype dependent upon p53 degradation. A single amino acid substitution at position Cys63 of SEQ ID NO:6 (=Cys70 in SEQ ID NO:5) destroys several HPV16 E6 functions: p53 degradation, E6TP-1 degradation, activation of telomerase, and, consequently, immortalization of primary epithelial cells.

Generally, antigens that may be used herein may be proteins or peptides that differ from the naturally-occurring proteins or peptides but yet retain the necessary epitopes for functional activity. An antigen may comprise, consist essentially of, or consist of an amino acid sequence that is at least about 90%, 95%, 96%, 97%, 98%, or 99% identical to that of the naturally-occurring antigen or a fragment thereof. An antigen may also comprise, consist essentially of, or consist of an amino acid sequence that is encoded by a nucleotide sequence that is at least about 90%, 95%, 96%, 97%, 98%, or 99% identical to a nucleotide sequence encoding the naturally-occurring antigen or a fragment thereof. An antigen may also comprise, consist essentially of, or consist of an amino acid sequence that is encoded by a nucleic acid that hybridizes under high stringency conditions to a nucleic acid encoding the naturally-occurring antigen or a fragment thereof. Hybridization conditions are further described herein.

An exemplary fusion protein comprises, consists essentially of, or consists of, an amino acid sequence that is at least about 90%, 95%, 96%, 97%, 98%, or 99% identical to that of a viral protein, such as E6 or E7, such as an E6 or E7 sequence provided herein. Where the E6 or E7 protein is a detox E6 or E7 protein, the amino acid sequence of the protein included in the fusion protein may comprise, consists essentially of, or consists of an amino acid sequence that is at least about 90%, 95%, 96%, 97%, 98%, or 99% identical to that of an E6 or E7 protein, wherein the amino acids that render the protein a “detox” protein are present.

Sequences of DNA Encoding the Immunogenicity-Potentiating Polypeptide CRT and its Homologues and Domains/Fragments

Calreticulin (CRT), a well-characterized ˜46 kDa protein was described briefly above, as were a number of its biological and biochemical activities. As used herein, “calreticulin” or “CRT” refers to polypeptides and nucleic acids molecules having substantial identity (defined herein) to the exemplary human CRT sequences as described herein or homologues thereof, such as rabbit and rat CRT—well-known in the art. A CRT polypeptide is a polypeptides comprising a sequence identical to or substantially identical (defined herein) to the amino acid sequence of CRT. An exemplary nucleotide and amino acid sequence for a CRT used in the present compositions and methods are presented below. The terms “calreticulin” or “CRT” encompass native proteins as well as recombinantly produced modified proteins that, when fused with an antigen (at the DNA or protein level) promote the induction of induce immune responses and, promote angiogenesis, including a CTL response. Thus, the terms “calreticulin” or “CRT” encompass homologues and allelic variants of human CRT, including variants of native proteins constructed by in vitro techniques, and proteins isolated from natural sources. The CRT polypeptides of the invention, and sequences encoding them, also include fusion proteins comprising non-CRT sequences, particularly MHC class I-binding peptides; and also further comprising other domains, e.g., epitope tags, enzyme cleavage recognition sequences, signal sequences, secretion signals and the like.

The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof. A polypeptide or peptide can be routinely screened for its activity as an ER chaperone using assays known in the art, such as those set forth in Example 1. While the invention is not limited by any particular mechanism of action, in vivo chaperones promote the correct folding and oligomerization of many glycoproteins in the ER, including the assembly of the MHC class I heterotrimeric molecule (heavy (H) chain, β2m, and peptide). They also retain incompletely assembled MHC class I heterotrimeric complexes in the ER (Hauri, FEBS Lett. 476:32-37, 2000).

The sequences of CRT, including human CRT, are well known in the art (McCauliffe, J. Clin. Invest. 86:332-5, 1990; Burns, Nature 367:476-80, 1994; Coppolino, Int. J. Biochem. Cell Biol. 30:553-8, 2000). A sequence of the CRT gene that includes the CRT coding sequence, appears as GenBank Accession No. NM 004343 and is SEQ ID NO:8, below. The portion of this sequence that encodes the CRT protein is residues 68-1319 (shown in upper case below). The stop codon ending this sequence is italicized/underscored.

| | |
|---|---|
| 1 | gtccgtactg cagagccgct gccggagggt cgttttaaag |
| | ggccgcgttg ccgccccctc |
| 61 | ggcccgccAT GCTGCTATCC GTGCCGCTGC TGCTCGGCCT |
| | CCTCGGCCTG GCCGTCGCCG |
| 121 | AGCCCGCCGT CTACTTCAAG GAGCAGTTTC TGGACGGAGA |
| | CGGGTGGACT TCCCGCTGGA |
| 181 | TCGAATCCAA ACACAAGTCA GATTTTGGCA AATTCGTTCT |
| | CAGTTCCGGC AAGTTCTACG |
| 241 | GTGACGAGGA GAAAGATAAA GGTTTGCAGA CAAGCCAGGA |
| | TGCACGCTTT TATGCTCTGT |
| 301 | CGGCCAGTTT CGAGCCTTTC AGCAACAAAG GCCAGACGCT |
| | GGTGGTGCAG TTCACGGTGA |
| 361 | AACATGAGCA GAACATCGAC TGTGGGGGCG GCTATGTGAA |
| | GCTGTTTCCT AATAGTTTGG |
| 421 | ACCAGACAGA CATGCACGGA GACTCAGAAT ACAACATCAT |
| | GTTTGGTCCC GACATCTGTG |
| 481 | GCCCTGGCAC CAAGAAGGTT CATGTCATCT TCAACTACAA |
| | GGGCAAGAAC GTGCTGATCA |
| 541 | ACAAGGACAT CCGTTGCAAG GATGATGAGT TTACACACCT |
| | GTACACACTG ATTGTGCGGC |
| 601 | CAGACAACAC CTATGAGGTG AAGATTGACA ACAGCCAGGT |
| | GGAGTCCGGC TCCTTGGAAG |
| 661 | ACGATTGGGA CTTCCTGCCA CCCAAGAAGA TAAAGGATCC |
| | TGATGCTTCA AAACCGGAAG |
| 721 | ACTGGGATGA GCGGGCCAAG ATCGATGATC CCACAGACTC |
| | CAAGCCTGAG GACTGGGACA |
| 781 | AGCCCGAGCA TATCCCTGAC CCTGATGCTA AGAAGCCCGA |
| | GGACTGGGAT GAAGAGATGG |
| 841 | ACGGAGAGTG GGAACCCCCA GTGATTCAGA ACCCTGAGTA |
| | CAAGGGTGAG TGGAAGCCCC |
| 901 | GGCAGATCGA CAACCCAGAT TACAAGGGCA CTTGGATCCA |
| | CCCAGAAATT GACAACCCCG |
| 961 | AGTATTCTCC CGATCCCAGT ATCTATGCCT ATGATAACTT |
| | TGGCGTGCTG GGCCTGGACC |
| 1021 | TCTGGCAGGT CAAGTCTGGC ACCATCTTTG ACAACTTCCT |
| | CATCACCAAC GATGAGGCAT |
| 1081 | ACGCTGAGGA GTTTGGCAAC GAGACGTGGG GCGTAACAAA |
| | GGCAGCAGAG AAACAAATGA |
| 1141 | AGGACAAACA GGACGAGGAG CAGAGGCTTA AGGAGGAGGA |
| | AGAAGACAAG AAACGCAAAG |
| 1201 | AGGAGGAGGA GGCAGAGGAC AAGGAGGATG ATGAGGACAA |
| | AGATGAGGAT GAGGAGGATG |
| 1261 | AGGAGGACAA GGAGGAAGAT GAGGAGGAAG ATGTCCCCGG |
| | CCAGGCCAAG GACGAGCTGt |
| 1321 | agagaggcct gcctccaggg ctggactgag gcctgagcgc |
| | tcctgccgca gagcttgccg |
| 1381 | cgccaaataa tgtctctgtg agactcgaga actttcattt |
| | ttttccaggc tggttcggat |
| 1441 | ttggggtgga ttttggtttt gttcccctcc tccactctcc |
| | cccaccccct ccccgccctt |
| 1501 | tttttttttt tttttaaact ggtattttat cctttgattc |
| | tccttcagcc ctcacccctg |
| 1561 | gttctcatct ttcttgatca acatcttttc ttgcctctgt |
| | gccccttctc tcatctctta |
| 1621 | gctcccctcc aacctggggg gcagtggtgt ggagaagcca |
| | caggcctgag atttcatctg |
| 1681 | ctctccttcc tggagcccag aggagggcag cagaaggggg |
| | tggtgtctcc aaccccccag |
| 1741 | cactgaggaa gaacggggct cttctcattt cacccctccc |
| | tttctcccct gcccccagga |
| 1801 | ctgggccact tctgggtggg gcagtgggtc ccagattggc |
| | tcacactgag aatgtaagaa |
| 1861 | ctacaaacaa aatttctatt aaattaaatt ttgtgtctc |
| | 1899 |

The human CRT coding sequence is shown below (SEQ ID NO:9):

| | |
|---|---|
| 1 | atgctgctat ccgtgccgct gctgctcggc ctcctcggcc |
| | tggccgtcgc cgagcccgcc |
| 61 | gtctacttca aggagcagtt tctggacgga gacgggtgga |
| | cttcccgctg gatcgaatcc |
| 121 | aaacacaagt cagattttgg caaattcgtt ctcagttccg |
| | gcaagttcta cggtgacgag |
| 181 | gagaaagata aaggtttgca gacaagccag gatgcacgct |
| | tttatgctct gtcggccagt |
| 241 | ttcgagcctt tcagcaacaa aggccagacg ctggtggtgc |
| | agttcacggt gaaacatgag |
| 301 | cagaacatcg actgtggggg cggctatgtg aagctgtttc |
| | ctaatagttt ggaccagaca |
| 361 | gacatgcacg gagactcaga atacaacatc atgtttggtc |
| | ccgacatctg tggccctggc |
| 421 | accaagaagg ttcatgtcat cttcaactac aagggcaaga |
| | acgtgctgat caacaaggac |
| 481 | atccgttgca aggatgatga gtttacacac ctgtacacac |
| | tgattgtgcg gccagacaac |
| 541 | acctatgagg tgaagattga caacagccag gtggagtccg |
| | gctccttgga agacgattgg |
| 601 | gacttcctgc cacccaagaa gataaaggat cctgatgctt |
| | caaaaccgga agactgggat |
| 661 | gagcgggcca agatcgatga tcccacagac tccaagcctg |
| | aggactggga caagcccgag |
| 721 | catatccctg accctgatgc taagaagccc gaggactggg |
| | atgaagagat ggacggagag |
| 781 | tgggaacccc cagtgattca gaaccctgag tacaagggtg |
| | agtggaagcc ccggcagatc |
| 841 | gacaacccag attacaaggg cacttggatc cacccagaaa |
| | ttgacaaccc cgagtattct |
| 901 | cccgatccca gtatctatgc ctatgataac tttggcgtgc |
| | tgggcctgga cctctggcag |
| 961 | gtcaagtctg gcaccatctt tgacaacttc ctcatcacca |
| | acgatgaggc atacgctgag |
| 1021 | gagtttggca acgagacgtg gggcgtaaca aaggcagcag |
| | agaaacaaat gaaggacaaa |
| 1081 | caggacgagg agcagaggct taaggaggag gaagaagaca |
| | agaaacgcaa agaggaggag |
| 1141 | gaggcagagg acaaggagga tgatgaggac aaagatgagg |
| | atgaggagga tgaggaggac |
| 1201 | aaggaggaag atgaggagga agatgtcccc ggccaggcca |
| | aggacgagct gtag 1251 |

The amino acid sequence of the human CRT protein (GenBank Accession No. NM 004343) (SEQ ID NO:10) is shown below:

| 1 MLLSVPLLLG LLGLAVAEPA VYFKEQFLDG DGWTSRWIES KHKSDFGKFV LSSGKFYGDE | |
|---|---|
| 61 EKDKGLQTSQ DARFYALSAS FEPFSNKGQT LVVQFTVKHE QNIDCGGGYV KLFPNSLDQT | |
| 121 DMHGDSEYNI MFGPDICGPG TKKVHVIFNY KGKNVLINKD IRCKDDEFTH LYTLIVRPDN | |
| 181 TYEVKIDNSQ VESGSLEDDW DFLPPKKIKD PDASKPEDWD ERAKIDDPTD SKPEDWDKPE | |
| 241 HIPDPDAKKP EDWDEEMDGE WEPPVIQNPE YKGEWKPRQI DNPDYKGTWI HPEIDNPEYS | |
| 301 PDPSIYAYDN FGVLGLDLWQ VKSGTIFDNF LITNDEAYAE EFGNETWGVT KAAEKQMKDK | |
| 361 QDEEQRLKEE EEDKKRKEEE EAEDKEDDED KDEDEEDEED KEEDEEEDVP GQAKDEL | 417 |

The amino acid sequence of the rabbit CRT protein (GenBank Accession No. P15253) (SEQ ID NO:11) is shown below

| 1 MLLPVPLLLG LLGLAAAEPV VYFKEQFLDG DGWTERWIES KHKSDFGKFV LSSGKFYGDQ | |
|---|---|
| 61 EKDKGLQTSQ DARFYALSAR FEPFSNKGQP LVVQFTVKHE QNIDCGGGYV KLFPAGLDQK | |
| 121 DMHGDSEYNI MFGPDICGPG TKKVHVIFNY KGKNVLINKD IRCKDDEFTH LYTLIVRPDN | |
| 181 TYEVKIDNSQ VESGSLEDDW DFLPPKKIKD PDASKPEDWD ERAKIDDPTD SKPEDWDKPE | |
| 241 HIPDPDAKKP EDWDEEMDGE WEPPVIQNPE YKGEWKPRQI DNPDYKGTWI HPEIDNPEYS | |
| 301 PDANIYAYDS FAVLGLDLWQ VKSGTIFDNF LITNDEAYAE EFGNETWGVT KTAEKQMKDK | |
| 361 QDEEQRLKEE EEEKKRKEEE EAEEDEEDKD DKEDEDEDEE DKDEEEEEAA AGQAKDEL | 418 |

The rabbit nucleotide sequence encoding CRT is shown below (SEQ ID NO:12) (including the stop codon which is underscored and not counted)

| 1 atgctgctcc ctgtgccgct gctgctcggc ctgctcggcc tggccgccgc cgagcccgtc | |
|---|---|
| 61 gtctacttca aggagcagtt tctggacgga gatgggtgga ccgagcgctg gatcgaatcc | |
| 121 aaacacaagt ccgattttgg caaattcgtc ctcagttcgg gcaagttcta cggcgatcag | |
| 181 gagaaagata aagggctgca gaccagccag gacgcccgct tctacgccct gtcggcccga | |
| 241 ttcgagccgt tcagcaacaa gggccagcca ctggtggtgc agttcaccgt gaaacacgag | |
| 301 cagaacattg actgcggggg cggctacgtg aagctgtttc cggccggcct ggaccagaag | |
| 361 gacatgcacg gggactctga gtacaacatc atgtttggtc ctgacatctg tggccccggc | |
| 421 accaagaagg ttcacgtcat cttcaactac aagggcaaga acgtgctgat caacaaggac | |
| 481 atccgttgca aggacgacga gttcacacac ctgtacacgc tgatcgtgcg gccggacaac | |
| 541 acgtatgagg tgaagattga caacagccag gtggagtcgg gctccctgga ggatgactgg | |
| 601 gacttcctac cccccaagaa gataaaggac ccagatgcct cgaagcctga agactgggac | |
| 661 gagcgggcca agatcgacga ccccacggac tccaagcccg aggactggga caagcccgag | |
| 721 cacatccccg acccggacgc gaagaagccc gaagactggg acgaagaaat ggacggagag | |
| 781 tgggagccgc cggtgattca gaaccccgag tacaagggtg agtggaagcc gcggcagatc | |
| 841 gacaaccccg attacaaagg cacctggatc caccccgaaa tcgacaaccc cgagtactcg | |
| 901 cccgacgcta acatctatgc ctacgacagc tttgccgtgc tgggcttgga cctctggcag | |
| 961 gtcaagtcgg gcaccatctt cgacaacttc ctcatcacca acgatgaggc gtacgcagag | |
| 1021 gagtttggca acgagacgtg gggcgtcacc aagacggccg agaagcagat gaaagacaag | |
| 1081 caggacgagg agcagcggct gaaggaggag gaggaggaga agaagcggaa ggaggaggag | |
| 1141 gaggccgagg aggacgagga ggacaaggac gacaaggagg acgaggatga ggacgaggag | |
| 1201 gacaaggacg aggaggagga ggaggcggcc gccggccagg ccaaggacga gctg tag | 1254 |

Rat CRT protein (GenBank Accession No. NM 022399) (SEQ ID NO:13) is shown below.

| 1 MLLSVPLLLG LLGLAAADPA IYFKEQFLDG DAWTNRWVES KHKSDFGKFV LSSGKFYGDQ | |
|---|---|
| 61 EKDKGLQTSQ DARFYALSAR FEPFSNKGQT LVVQFTVKHE QNIDCGGGYV KLFPGGLDQK | |
| 121 DMHGDSEYNI MFGPDICGPG TKKVHVIFNY KGKNVLINKD IRCKDDEFTH LYTLIVRPDN | |
| 181 TYEVKIDNSQ VESGSLEDDW DFLPPKKIKD PDAAKPEDWD ERAKIDDPTD SKPEDWDKPE | |
| 241 HIPDPDAKKP EDWDEEMDGE WEPPVIQNPE YKGEWKPRQI DNPDYKGTWI HPEIDNPEYS | |
| 301 PDANIYAYDS FAVLGLDLWQ VKSGTIFDNF LITNDEAYAE EFGNETWGVT KAAEKQMKDK | |
| 361 QDEEQRLKEE EEDKKRKEEE EAEDKEDEDD RDEDEDEEDE KEEDEEDATG QAKDEL | 416 |

The corresponding coding sequence is not shown for the sake of brevity.

Table 1 compares aligned sequences of human, rabbit and rat CRT (SEQ ID NO:11, 12, and 13), respectively. The changes from human to rabbit and human to rat are underscored. As stated above, these proteins are highly conserved, and most of the amino acid differences between species are conservative in nature. Most of the variation is found in the alignment of the approximately 36 C-terminal residues. Thus, for the present invention, although human CRT is preferred, DNA encoding any homologue of CRT from any species that has the requisite biological activity (as an IPP) or any active domain or fragment thereof, may be used in place of human CRT or a domain thereof.

TABLE 1
Sequence Alignment of Human, Rabbit and Rat* CRT Polypeptides

| H | 1 MLLSVPLLLG LLGLAVAEPA VYFKEQFLDG DGWTSRWIES KHKSDFGKFV LSSGKFYGDE | |
|---|---|---|
| Rb | 1 MLLPVPLLLG LLGLAAAEPV VYFKEQFLDG DGWTERWIES KHKSDFGKFV LSSGKFYGDQ | |
| Rt | 1 MLLSVPLLLG LLGLAAADPA IYFKEQFLDG DAWTNRWVES KHKSDFGKFV LSSGKFYGDQ | |
| H | 61 EKDKGLQTSQ DARFYALSAS FEPFSNKGQT LVVQFTVKHE QNIDCGGGYV KLFPNSLDQT | |
| Rb | 61 EKDKGLQTSQ DARFYALSAR FEPFSNKGQP LVVQFTVKHE QNIDCGGGYV KLFPAGLDQK | |
| Rt | 61 EKDKGLQTSQ DARFYALSAR FEPFSNKGQT LVVQFTVKHE QNIDCGGGYV KLFPGGLDQK | |
| H | 121 DMHGDSEYNI MFGPDICGPG TKKVHVIFNY KGKNVLINKD IRCKDDEFTH LYTLIVRPDN | |
| Rb | 121 DMHGDSEYNI MFGPDICGPG TKKVHVIFNY KGKNVLINKD IRCKDDEFTH LYTLIVRPDN | |
| Rt | 121 DNHGDSEYNI MFGPDICGPG TKKVHVIFNY KGKNVLINKD IRCKDDEFTH LYTLIVRPDN | |
| H | 181 TYEVKIDNSQ VESGSLEDDW DFLPPKKIKD PDASKPEDWD ERAKIDDPTD SKPEDWDKPE | |
| Rb | 181 TYEVKIDNSQ VESGSLEDDW DFLPPKKIKD PDASKPEDWD ERAKIDDPTD SKPEDWDKPE | |
| Rt | 181 TYEVKIDNSQ VESGSLEDDW DFLPPKKIKD PDAAKPEDWD ERAKIDDPTD SKPEDWDKPE | |
| H | 241 HIPDPDAKKP EDWDEEMDGE WEPPVIQNPE YKGEWKPRQI DNPDYKGTWI HPEIDNPEYS | |
| Rb | 241 HIPDPDAKKP EDWDEEMDGE WEPPVIQNPE YKGEWKPRQI DNPDYKGTWI HPEIDNPEYS | |
| Rt | 241 HIPDPDAKKP EDWDEEMDGE WEPPVIQNPE YKGEWKPRQI DNPDYKGTWI HPEIDNPEYS | |
| H | 301 PDPSIYAYDN FGVLGLDLWQ VKSGTIFDNF LITNDEAYAE EFGNETWGVT KAAEKQMKDK | |
| Rb | 301 PDANIYAYDS FAVLGLDLWQ VKSGTIFDNF LITNDEAYAE EFGNETWGVT KTAEKQMKDK | |
| Rt | 301 PDANIYAYDS FAVLGLDLWQ VKSGTIFDNF LITNDEAYAE EFGNETWGVT KAAEKQMKDK | |
| H | 361 QDEEQRLKEE EEDKKRKEEE EAEDKEDDED K DEDEEDEED KEEDEEEDVPG QAKDEL | 417 |
| Rb | 361 QDEEQRLKEE EEEKKRKEEE EAEEDEEDKDDKEDEDE_DEED KDEEEEEAAAG QAKDEL | 418 |
| Rt | 361 QDEEQRLKEE EEDKKRKEEE EAEDKEDEDD R DEDE_DEEDE KEEDEE_DATG QAKDEL | 416 |

*H: human; Rb: rabbint; Rt: rat

CRT Domains

The present inventors and colleagues (Cheng et al., supra; incorporated by reference in its entirety) that DNA vaccines encoding each of the N, P, and C domains of CRT chimerically linked to HPV-16 E7 elicited potent antigen-specific CD8+ T cell responses and antitumor immunity in mice vaccinated i.d., by gene gun administration. N-CRT/E7, P-CRT/E7 or C-CRT/E7 DNA each exhibited significantly increased numbers of E7-specific CD8⁺ T cell precursors and impressive antitumor effects against E7-expressing tumors when compared with mice vaccinated with E7 DNA (antigen only). N-CRT DNA administration also resulted in anti-angiogenic antitumor effects. Thus, cancer therapy using DNE encoding N-CRT linked to a tumor antigen may be used for treating tumors through a combination of antigen-specific immunotherapy and inhibition of angiogenesis.

The constructs comprising CRT or one of its domains linked to E7 is illustrated schematically below.

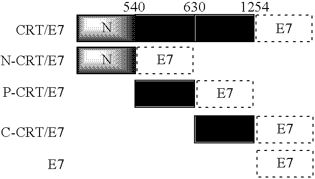

The amino acid sequences of the 3 human CRT domains are shown as annotations of the full length protein (SEQ. ID NO:10). The N domain comprises residues 1-170 (normal text); the P domain comprises residues 171-269 (underscored); and the C domain comprises residues 270-417 (bold/italic)

| 1 MLLSVPLLLG LLGLAVAEPA VYFKEQFLDG DGWTSRWIES KHKSDFGKFV LSSGKFYGDE | |
|---|---|
| 61 EKDKGLQTSQ DARFYALSAS FEPFSNKGQT LVVQFTVKHE QNIDCGGGYV KLFPNSLDQT | |
| 121 DMHGDSEYNI MFGPDICGPG TKKVHVIFNY KGKNVLINKD IRCKDDEFTH LYTLIVRPDN | |
| 181 TYEVKIDNSQ VESGSLEDDW DFLPPKKIKD PDASKPEDWD ERAKIDDPTD SKPEDWDKPE | |
| 241 HIPDPDAKKP EDWDEEMDGE WEPPVIQNPE YKGEWKPRQ [symbol] | |
| 301 [symbol] | |
| 361 [symbol] | 417 |

The sequences of the three domains are shown as separate polypeptides below:

| Human N-CRT | |
|---|---|
| (SEQ ID NO:14) | |
| 1 MLLSVPLLLG LLGLAVAEPA VYFKEQFLDG DGWTSRWIES KHKSDFGKFV LSSGKFYGDE | |
| 61 EKDKGLQTSQ DARFYALSAS FEPFSNKGQT LVVQFTVKHE QNIDCGGGYV KLFPNSLDQT | |
| 121 DMHGDSEYNI MFGPDICGPG TKKVHVIFNY KGKNVLINKD IRCKDDEFTH | 170 |
| Human P-CRT | |
| (SEQ ID NO:15) | |
| 1 LYTLIVRPDN TYEVKIDNSQ VESGSLEDDW DFLPPKKIKD PDASKPEDWD ERAKIDDPTD | |
| 61 SKPEDWDKPE HIPDPDAKKP EDWDEEMDGE WEPPVIQNPE YKGEWKPRQ | 109 |
| Human C-CRT | |
| (SEQ ID NO:16) | |
| 1 IDNPDYKGTW IHPEIDNPEY SPDPSIYAYD NFGVLGLDLW QVKSGTIFDN FLITNDEAYA | |
| 61 EEFGNETWGV TKAAEKQMKD KQDEEQRLKE EEEDKKRKEE EEAEDKEDDE DKDEDEEDEE | |
| 121 DKEEDEEEDV PGQAKDEL | 138 |

The present vectors may comprises DNA encoding one or more of these domain sequences, which are shown by annotation of SEQ ID NO:9, below, wherein the N-domain sequence is upper case, the P-domain sequence is lower case/italic/underscored, and the C domain sequence is lower case. The stop codon is also shown but not counted.

| 1 ATGCTGCTAT CCGTGCCGCT GCTGCTCGGC CTCCTCGGCC TGGCCGTCGC CGAGCCCGCC | |
|---|---|
| 61 GTCTACTTCA AGGAGCAGTT TCTGGACGGA GACGGGTGGA CTTCCCGCTG GATCGAATCC | |
| 121 AAACACAAGT CAGATTTTGG CAAATTCGTT CTCAGTTCCG GCAAGTTCTA CGGTGACGAG | |
| 181 GAGAAAGATA AAGGTTTGCA GACAAGCCAG GATGCACGCT TTTATGCTCT GTCGGCCAGT | |
| 241 TTCGAGCCTT TCAGCAACAA AGGCCAGACG CTGGTGGTGC AGTTCACGGT GAAACATGAG | |
| 301 CAGAACATCG ACTGTGGGGG CGGCTATGTG AAGCTGTTTC CTAATAGTTT GGACCAGACA | |
| 361 GACATGCACG GAGACTCAGA ATACAACATC ATGTTTGGTC CCGACATCTG TGGCCCTGGC | |
| 421 ACCAAGAAGG TTCATGTCAT CTTCAACTAC AAGGGCAAGA ACGTGCTGAT CAACAAGGAC | |
| 481 ATCCGTTGCA AGGATGATGA GTTTACACAC CTGTACACAC TGATTGTGCG GCCAGACAAC | |
| 541 acctatgagg tgaagattga caacagccag gtggagtccg gctccttgga agacgattgg | |
| 601 gacttcctgc cacccaagaa gataaaggat cctgatgctt caaaaccgga agactgggat | |
| 661 gagcgggcca agatcgatga tcccacagac tccaagcctg aggactggga caagcccgag | |
| 721 catatccctg accctgatgc taagaagccc gaggactggg atgaagagat ggacggagag | |
| 781 tgggaacccc cagtgattca gaaccctgag tacaagggtg agtggaagcc ccggcagatc | |
| 841 gacaacccag attacaaggg cacttggatc cacccagaaa ttgacaaccc cgagtattct | |
| 901 cccgatccca gtatctatgc ctatgataac tttggcgtgc tgggcctgga cctctggcag | |
| 961 gtcaagtctg gcaccatctt tgacaacttc ctcatcacca acgatgaggc atacgctgag | |
| 1021 gagtttggca acgagacgtg gggcgtaaca aaggcagcag agaaacaaat gaaggacaaa | |
| 1081 caggacgagg agcagaggct taaggaggag gaagaagaca agaaacgcaa agaggaggag | |
| 1141 gaggcagagg acaaggagga tgatgaggac aaagatgagg atgaggagga tgaggaggac | |
| 1201 aaggaggaag atgaggagga agatgtcccc ggccaggcca aggacgagct gtag | 1251 |

The coding sequence for each separate domain is provided below:

| Human N-CRT DNA | |
|---|---|
| (SEQ ID NO:17) | |
| 1 ATGCTGCTAT CCGTGCCGCT GCTGCTCGGC CTCCTCGGCC TGGCCGTCGC CGAGCCCGCC | |
| 61 GTCTACTTCA AGGAGCAGTT TCTGGACGGA GACGGGTGGA CTTCCCGCTG GATCGAATCC | |
| 121 AAACACAAGT CAGATTTTGG CAAATTCGTT CTCAGTTCCG GCAAGTTCTA CGGTGACGAG | |
| 181 GAGAAAGATA AAGGTTTGCA GACAAGCCAG GATGCACGCT TTTATGCTCT GTCGGCCAGT | |
| 241 TTCGAGCCTT TCAGCAACAA AGGCCAGACG CTGGTGGTGC AGTTCACGGT GAAACATGAG | |
| 301 CAGAACATCG ACTGTGGGGG CGGCTATGTG AAGCTGTTTC CTAATAGTTT GGACCAGACA | |
| 361 GACATGCACG GAGACTCAGA ATACAACATC ATGTTTGGTC CCGACATCTG TGGCCCTGGC | |
| 421 ACCAAGAAGG TTCATGTCAT CTTCAACTAC AAGGGCAAGA ACGTGCTGAT CAACAAGGAC | |
| 481 ATCCGTTGCA AGGATGATGA GTTTACACAC CTGTACACAC TGATTGTGCG GCCAGACAAC | |
| Human P-CRT DNA | |
| (SEQ ID NO:18) | |
| 1 acctatgagg tgaagattga caacagccag gtggagtccg gctccttgga agacgattgg | |
| 61 gacttcctgc cacccaagaa gataaaggat cctgatgctt caaaaccgga agactgggat | |
| 121 gagcgggcca agatcgatga tcccacagac tccaagcctg aggactggga caagcccgag | |
| 181 catatccctg accctgatgc taagaagccc gaggactggg atgaagagat ggacggagag | |
| 241 tgggaacccc cagtgattca gaaccct | 267 |
| Human C-CRT DNA | |
| (SEQ ID NO:19) | |
| 1 gagtacaagg gtgagtggaa gccccggcag atcgacaacc cagattacaa gggcacttgg | |
| 61 atccacccag aaattgacaa ccccgagtat tctcccgatc ccagtatcta tgcctatgat | |
| 121 aactttggcg tgctgggcct ggacctctgg caggtcaagt ctggcaccat ctttgacaac | |
| 181 ttcctcatca ccaacgatga ggcatacgct gaggagtttg gcaacgagac gtggggcgta | |
| 241 acaaaggcag cagagaaaca aatgaaggac aaacaggacg aggagcagag gcttaaggag | |
| 301 gaggaagaag acaagaaacg caaagaggag gaggaggcag aggacaagga ggatgatgag | |
| 361 gacaaagatg aggatgagga ggatgaggag gacaaggagg aagatgagga ggaagatgtc | |
| 421 cccggccagg ccaaggacga gctg | 444 |

Alternatively, any nucleotide sequences that encodes these domains may be used in the present constructs. Thus, for use in humans, the sequences may be further codon-optimized.

The present construct may employ combinations of one or more CRT domains, in any of a number of orientations. Using the designations N^{CRT}, P^{CRT} and C^{CRT} to designate the domains, the following are but a few examples of the combinations that may be used in the DNA vaccine vectors of the present invention (where it is understood that Ag can be any antigen, preferably E7(detox) or E6 (detox). N^{CRT}-P^{CRT}-Ag; N^{CRT}-P^{CRT}-Ag; N^{CRT}-C^{CRT}-Ag; N^{CRT}-N^{CRT}-Ag; N^{CRT}-N^{CRT}-N^{CRT}-Ag; P^{CRT}-P^{CRT}-AG; P^{CRT}-C^{CRT}-Ag; P^{CRT}-N^{CRT}-Ag; C^{CRT}-P^{CRT}-Ag; N^{CRT}-P^{CRT}-Ag; etc.

The present invention may employ shorter polypeptide fragments of CRT or CRT domains provided such fragments can enhance the immune response to an antigen with which they are paired. Shorter peptides from the CRT or domain sequences shown above that have the ability to promote protein processing via the MHC-1 class I pathway are also included, and may be defined by routine experimentation.

The present invention may also employ shorter nucleic acid fragments that encode CRT or CRT domains provided such fragments are functional, e.g., encode polypeptides that can enhance the immune response to an antigen with which they are paired (e.g., linked). Nucleic acids that encode shorter peptides from the CRT or domain sequences shown above and are functional, e.g., have the ability to promote protein processing via the MHC-1 class I pathway, are also included, and may be defined by routine experimentation.

A polypeptide fragment of CRT may include at least or about 50, 100, 200, 300, or 400 amino acids. A polypeptide fragment of CRT may also include at least or about 25, 50, 75, 100, 25-50, 50-100, or 75-125 amino acids from a CRT domain selected from the group consisting of the N-CRT, P-CRT, and C-CRT. A polypeptide fragment of CRT may include residues 1-50, 50-75, 75-100, 100-125, 125-150, 150-170 of the N-domain (e.g., of SEQ ID NO: 14). A polypeptide fragment of CRT may include residues 1-50, 50-75, 75-100, 100-109 of the P-domain (e.g., of SEQ ID NO: 15). A polypeptide fragment of CRT may include residues 1-50, 50-75, 75-100, 100-125, 125-138 of the C-domain (e.g., of SEQ ID NO: 16).

A nucleic acid fragment of CRT may encode at least or about 50, 100, 200, 300, or 400 amino acids. A nucleic acid fragment of CRT may also encode at least or about 25, 50, 75, 100, 25-50, 50-100, or 75-125 amino acids from a CRT domain selected from the group consisting of the N-CRT, P-CRT, and C-CRT. A nucleic acid fragment of CRT may encode residues 1-50, 50-75, 75-100, 100-125, 125-150, 150-170 of the N-domain (e.g., of SEQ ID NO: 14). A nucleic acid fragment of CRT may encode residues 1-50, 50-75, 75-100, 100-109 of the P-domain (e.g., of SEQ ID NO: 15). A nucleic acid fragment of CRT may encode residues 1-50, 50-75, 75-100, 100-125, 125-138 of the C-domain (e.g., of SEQ ID NO: 16).

Polypeptide “fragments” of CRT, as provided herein, do not include full-length CRT. Likewise, nucleic acid “fragments” of CRT, as provided herein, do not include a full-length CRT nucleic acid sequence and do not encode a full-length CRT polypeptide.

A most preferred vector construct of a complete chimeric nucleic acid of the invention, is shown below (SEQ ID NO:20). The sequence is annotated to show plasmid-derived nucleotides (lower case letters), CRT-derived nucleotides (upper case bold letters), and HPV-E7-derived nucleotides (upper case, italicized/underlined letters). Note that 5 plasmid nucleotides are found between the CRT and E7 coding sequences and that the stop codon for the E7 sequence is double underscored. This plasmid is also referred to as pNGVL4a-CRT/E7(detox).

| 1 gctccgcccc cctgacgagc atcacaaaaa tcgacgctca agtcagaggt ggcgaaaccc | |
|---|---|
| 61 gacaggacta taaagatacc aggcgtttcc ccctggaagc tccctcgtgc gctctcctgt | |
| 121 tccgaccctg ccgcttaccg gatacctgtc cgcctttctc ccttcgggaa gcgtggcgct | |
| 181 ttctcatagc tcacgctgta ggtatctcag ttcggtgtag gtcgttcgct ccaagctggg | |
| 241 ctgtgtgcac gaaccccccg ttcagcccga ccgctgcgcc ttatccggta actatcgtct | |
| 301 tgagtccaac ccggtaagac acgacttatc gccactggca gcagccactg gtaacaggat | |
| 361 tagcagagcg aggtatgtag gcggtgctac agagttcttg aagtggtggc ctaactacgg | |
| 421 ctacactaga agaacagtat ttggtatctg cgctctgctg aagccagtta ccttcggaaa | |
| 481 aagagttggt agctcttgat ccggcaaaca aaccaccgct ggtagcggtg gtttttttgt | |
| 541 ttgcaagcag cagattacgc gcagaaaaaa aggatctcaa gaagatcctt tgatcttttc | |
| 601 tacggggtct gacgctcagt ggaacgaaaa ctcacgttaa gggattttgg tcatgagatt | |
| 661 atcaaaaagg atcttcacct agatcctttt aaattaaaaa tgaagtttta aatcaatcta | |
| 721 aagtatatat gagtaaactt ggtctgacag ttaccaatgc ttaatcagtg aggcacctat | |
| 781 ctcagcgatc tgtctatttc gttcatccat agttgcctga ctcggggggg gggggcgctg | |
| 841 aggtctgcct cgtgaagaag gtgttgctga ctcataccag ggcaacgttg ttgccattgc | |
| 901 tacaggcatc gtggtgtcac gctcgtcgtt tggtatggct tcattcagct ccggttccca | |
| 961 acgatcaagg cgagttacat gatcccccat gttgtgcaaa aaagcggtta gctccttcgg | |
| 1021 tcctccgatc gttgtcagaa gtaagttggc cgcagtgtta tcactcatgg ttatggcagc | |
| 1081 actgcataat tctcttactg tcatgccatc cgtaagatgc ttttctgtga ctggtgagta | |
| 1141 ctcaaccaag tcattctgag aatagtgtat gcggcgaccg agttgctctt gcccggcgtc | |
| 1201 aatacgggat aataccgcgc cacatagcag aactttaaaa gtgctcatca ttggaaaacg | |
| 1261 ttcttcgggg cgaaaactct caaggatctt accgctgttg agatccagtt cgatgtaacc | |
| 1321 cactcgtgca cctgaatcgc cccatcatcc agccagaaag tgagggagcc acggttgatg | |
| 1381 agagctttgt tgtaggtgga ccagttggtg attttgaact tttgctttgc cacggaacgg | |
| 1441 tctgcgttgt cgggaagatg cgtgatctga tccttcaact cagcaaaagt tcgatttatt | |
| 1501 caacaaagcc gccgtcccgt caagtcagcg taatgctctg ccagtgttac aaccaattaa | |
| 1561 ccaattctga ttagaaaaac tcatcgagca tcaaatgaaa ctgcaattta ttcatatcag | |
| 1621 gattatcaat accatatttt tgaaaaagcc gtttctgtaa tgaaggagaa aactcaccga | |
| 1681 ggcagttcca taggatggca agatcctggt atcggtctgc gattccgact cgtccaacat | |
| 1741 caatacaacc tattaatttc ccctcgtcaa aaataaggtt atcaagtgag aaatcaccat | |
| 1801 gagtgacgac tgaatccggt gagaatggca aaagcttatg catttctttc cagacttgtt | |
| 1861 caacaggcca gccattacgc tcgtcatcaa aatcactcgc atcaaccaaa ccgttattca | |
| 1921 ttcgtgattg cgcctgagcg agacgaaata cgcgatcgct gttaaaagga caattacaaa | |
| 1981 caggaatcga atgcaaccgg cgcaggaaca ctgccagcgc atcaacaata ttttcacctg | |
| 2041 aatcaggata ttcttctaat acctggaatg ctgttttccc ggggatcgca gtggtgagta | |
| 2101 accatgcatc atcaggagta cggataaaat gcttgatggt cggaagaggc ataaattccg | |
| 2161 tcagccagtt tagtctgacc atctcatctg taacatcatt ggcaacgcta cctttgccat | |
| 2221 gtttcagaaa caactctggc gcatcgggct tcccatacaa tcgatagatt gtcgcacctg | |
| 2281 attgcccgac attatcgcga gcccatttat acccatataa atcagcatcc atgttggaat | |
| 2341 ttaatcgcgg cctcgagcaa gacgtttccc gttgaatatg gctcataaca ccccttgtat | |
| 2401 tactgtttat gtaagcagac agttttattg ttcatgatga tatattttta tcttgtgcaa | |
| 2461 tgtaacatca gagattttga gacacaacgt ggctttcccc ccccccccat tattgaagca | |
| 2521 tttatcaggg ttattgtctc atgagcggat acatatttga atgtatttag aaaaataaac | |
| 2581 aaataggggt tccgcgcaca tttccccgaa aagtgccacc tgacgtctaa gaaaccatta | |
| 2641 ttatcatgac attaacctat aaaaataggc gtatcacgag gccctttcgt ctcgcgcgtt | |
| 2701 tcggtgatga cggtgaaaac ctctgacaca tgcagctccc ggagacggtc acagcttgtc | |
| 2761 tgtaagcgga tgccgggagc agacaagccc gtcagggcgc gtcagcgggt gttggcgggt | |
| 2821 gtcggggctg gcttaactat gcggcatcag agcagattgt actgagagtg caccatatgc | |
| 2881 ggtgtgaaat accgcacaga tgcgtaagga gaaaataccg catcagattg gctattggcc | |
| 2941 attgcatacg ttgtatccat atcataatat gtacatttat attggctcat gtccaacatt | |
| 3001 accgccatgt tgacattgat tattgactag ttattaatag taatcaatta cggggtcatt | |
| 3061 agttcatagc ccatatatgg agttccgcgt tacataactt acggtaaatg gcccgcctgg | |
| 3121 ctgaccgccc aacgaccccc gcccattgac gtcaataatg acgtatgttc ccatagtaac | |
| 3181 gccaataggg actttccatt gacgtcaatg ggtggagtat ttacggtaaa ctgcccactt | |
| 3241 ggcagtacat caagtgtatc atatgccaag tacgccccct attgacgtca atgacggtaa | |
| 3301 atggcccgcc tggcattatg cccagtacat gaccttatgg gactttccta cttggcagta | |
| 3361 catctacgta ttagtcatcg ctattaccat ggtgatgcgg ttttggcagt acatcaatgg | |
| 3421 gcgtggatag cggtttgact cacggggatt tccaagtctc caccccattg acgtcaatgg | |
| 3481 gagtttgttt tggcaccaaa atcaacggga ctttccaaaa tgtcgtaaca actccgcccc | |
| 3541 attgacgcaa atgggcggta ggcgtgtacg gtgggaggtc tatataagca gagctcgttt | |
| 3601 agtgaaccgt cagatcgcct ggagacgcca tccacgctgt tttgacctcc atagaagaca | |
| 3661 ccgggaccga tccagcctcc gcggccggga acggtgcatt ggaacgcgga ttccccgtgc | |
| 3721 caagagtgac gtaagtaccg cctatagact ctataggcac acccctttgg ctcttatgca | |
| 3781 tgctatactg tttttggctt ggggcctata cacccccgct tccttatgct ataggtgatg | |
| 3841 gtatagctta gcctataggt gtgggttatt gaccattatt gaccactcca acggtggagg | |
| 3901 gcagtgtagt ctgagcagta ctcgttgctg ccgcgcgcgc caccagacat aatagctgac | |
| 3961 agactaacag actgttcctt tccatgggtc ttttctgcag tcaccgtcgt cgacATGCTG | |
| 4021 CTATCCGTGC CGCTGCTGCT CGGCCTCCTC GGCCTGGCCG TCGCCGAGCC TGCCGTCTAC | |
| 4081 TTCAAGGAGC AGTTTCTGGA CGGGGACGGG TGGACTTCCC GCTGGATCGA ATCCAAACAC | |
| 4141 AAGTCAGATT TTGGCAAATT CGTTCTCAGT TCCGGCAAGT TCTACGGTGA CGAGGAGAAA | |
| 4201 GATAAAGGTT TGCAGACAAG CCAGGATGCA CGCTTTTATG CTCTGTCGGC CAGTTTCGAG | |
| 4261 CCTTTCAGCA ACAAAGGCCA GACGCTGGTG GTGCAGTTCA CGGTGAAACA TGAGCAGAAC | |
| 4321 ATCGACTGTG GGGGCGGCTA TGTGAAGCTG TTTCCTAATA GTTTGGACCA GACAGACATG | |
| 4381 CACGGAGACT CAGAATACAA CATCATGTTT GGTCCCGACA TCTGTGGCCC TGGCACCAAG | |
| 4441 AAGGTTCATG TCATCTTCAA CTACAAGGGC AAGAACGTGC TGATCAACAA GGACATCCGT | |
| 4501 TGCAAGGATG ATGAGTTTAC ACACCTGTAC ACACTGATTG TGCGGCCAGA CAACACCTAT | |
| 4561 GAGGTGAAGA TTGACAACAG CCAGGTGGAG TCCGGCTCCT TGGAAGACGA TTGGGACTTC | |
| 4621 CTGCCACCCA AGAAGATAAA GGATCCTGAT GCTTCAAAAC CGGAAGACTG GGATGAGCGG | |
| 4681 GCCAAGATCG ATGATCCCAC AGACTCCAAG CCTGAGGACT GGGACAAGCC CGAGCATATC | |
| 4741 CCTGACCCTG ATGCTAAGAA GCCCGAGGAC TGGGATGAAG AGATGGACGG AGAGTGGGAA | |
| 4801 CCCCCAGTGA TTCAGAACCC TGAGTACAAG GGTGAGTGGA AGCCCCGGCA GATCGACAAC | |
| 4861 CCAGATTACA AGGGCACTTG GATCCACCCA GAAATTGACA ACCCCGAGTA TTCTCCCGAT | |
| 4921 CCCAGTATCT ATGCCTATGA TAACTTTGGC GTGCTGGGCC TGGACCTCTG GCAGGTCAAG | |
| 4981 TCTGGCACCA TCTTTGACAA CTTCCTCATC ACCAACGATG AGGCATACGC TGAGGAGTTT | |
| 5041 GGCAACGAGA CGTGGGGCGT AACAAAGGCA GCAGAGAAAC AAATGAAGGA CAAACAGGAC | |
| 5101 GAGGAGCAGA GGCTTAAGGA GGAGGAAGAA GACAAGAAAC GCAAAGAGGA GGAGGAGGCA | |
| 5161 GAGGACAAGG AGGATGATGA GGACAAAGAT GAGGATGAGG AGGATGAGGA GGACAAGGAG | |
| 5221 GAAGATGAGG AGGAAGATGT CCCCGGCCAG GCCAAGGACG AGCTG [symbol] | |
| 5281 [symbol] | |
| 5341 [symbol] | |
| 5401 [symbol] | |
| 5461 [symbol] | |
| 5521 [symbol] AAgg atccagatct | |
| 5581 ttttccctct gccaaaaatt atggggacat catgaagccc cttgagcatc tgacttctgg | |
| 5641 ctaataaagg aaatttattt tcattgcaat agtgtgttgg aattttttgt gtctctcact | |
| 5701 cggaaggaca tatgggaggg caaatcattt aaaacatcag aatgagtatt tggtttagag | |
| 5761 tttggcaaca tatgcccatt cttccgcttc ctcgctcact gactcgctgc gctcggtcgt | |
| 5821 tcggctgcgg cgagcggtat cagctcactc aaaggcggta atacggttat ccacagaatc | |
| 5881 aggggataac gcaggaaaga acatgtgagc aaaaggccag caaaaggcca ggaaccgtaa | |
| 5941 aaaggccgcg ttgctggcgt ttttccatag | 5970 |

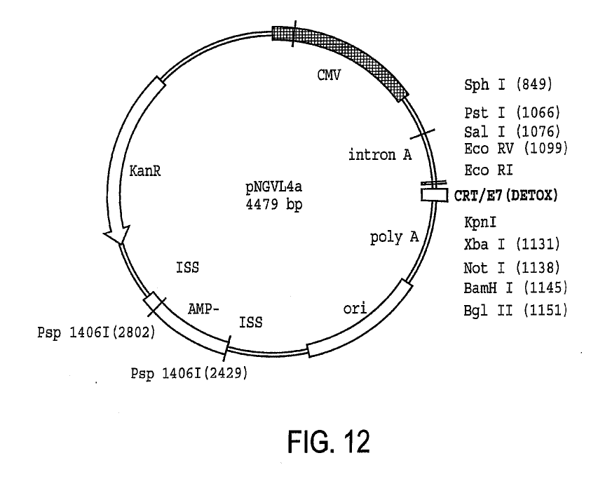

Table 2 below describes the structure of the above plasmid (see also FIG. 12 for diagram).

TABLE 2

| Plasmid Position | Genetic Construct | Source of Construct |
|---|---|---|
| 5970-0823 | E. coli ORI (ColEI) | pBR/E. coli -derived |
| 0837-0881 | portion of transposase (tpnA) | Common plasmid sequence Tn5/Tn903 |
| 0882-1332 | β-Lactamase (Amp^{R}) | pBRpUC derived plasmid |
| 1331-2496 | AphA (Kan^{R}) | Tn903 |
| 2509-2691 | P3 Promoter DNA binding site | Tn3/pBR322 |
| 2692-2926 | pUC backbone | Common plasmid sequence |
| | | pBR322-derived |
| 2931-4009 | NF1 binding and promoter | HHV-5(HCMV UL-10 IE1 gene) |
| 4010-4014 | Poly-cloning site | Common plasmid sequence |
| 4015-5265 | Calreticulin (CRT) | Human Calreticulin |
| 5266-5271 | GAATTC plasmid sequence | Remain after cloning |
| 5272-5568 | dE7 gene (detoxified partial) | HPV-16 (E7 gene) incl. stop codon |
| 5569-5580 | Poly-cloning site | Common plasmid sequence |
| 551-5970 | Poly-Adenylation site | Mammalian signal, pHCMV-derived |

Homologues or variants of CRT's as described herein, may also be used, provided that they have the requisite biological activity. These include various substitutions, deletions, or additions of the amino acid or nucleic acid sequences. Due to code degeneracy, for example, there may be considerable variation in nucleotide sequences encoding the same amino acid sequence.

A functional derivative of CRT retains measurable CRT-like activity, preferably that of promoting immunogenicity of one or more antigenic epitopes fused thereto by promoting presentation by class I pathways. “Functional derivatives” encompass “variants” and “fragments” regardless of whether the terms are used in the conjunctive or the alternative herein.

The term “chimeric” or “fusion” polypeptide or protein refers to a composition comprising at least one polypeptide or peptide sequence or domain that is chemically bound in a linear fashion with a second polypeptide or peptide domain. One embodiment of this invention is an isolated or recombinant nucleic acid molecule encoding a fusion protein comprising at least two domains, wherein the first domain comprises an IPP and the second domain comprises an antigenic epitope, e.g., an MHC class I-binding peptide epitope. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules of the invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of an IPP, preferably CRT (or of its coding sequence) which refers to an amino acid substitution variant, a “fragment,” etc., of the protein, which terms are defined below. A functional derivative of an IPP retains measurable activity, preferably that is manifest as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned.

In a preferred embodiment, the length of a sequence being compared is at least 30%, preferably at least 40%, more preferably at least 50%, even more preferably at least 60%, and even more preferably at least 70%, 80%, 90%, 95%, 96%, 97%, 98%, or 99% of the length of the reference sequence (e.g., CRT, SEQ ID NO:10). The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to CRT nucleic acid molecules. BLAST protein searches can be performed with the XBLAST program, score=50, wordlength=3 to obtain amino acid sequences homologous to HVP22 protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of CRT or of a CRT domain described above is characterized as having (a) functional activity of native CRT or domain thereof and (b) sequence similarity to a native CRT protein (such as SEQ ID NO:9) or domain thereof when determined as above, of at least about 20% (at the amino acid level), preferably at least about 40%, more preferably at least about 70%, more preferably at least about 90%, 95%, 96%, 97%, 98%, or 99%.

It is within the skill in the art to obtain and express such a protein using DNA probes based on the disclosed sequences of CRT. Then, the fusion protein's biochemical and biological activity can be tested readily using art-recognized methods such as those described herein, for example, a T cell proliferation, cytokine secretion or a cytolytic assay, or an in vivo assay of tumor protection or tumor therapy. A biological assay of the stimulation of antigen-specific T cell reactivity will indicate whether the homologue has the requisite activity to qualify as a “functional” homologue.

A “variant” refers to a molecule substantially identical to either the full protein or to a fragment thereof in which one or more amino acid residues have been replaced (substitution variant) or which has one or several residues deleted (deletion variant) or added (addition variant). A “fragment” of CRT refers to any subset of the molecule, that is, a shorter polypeptide of the full-length protein.

A number of processes can be used to generate fragments, mutants and variants of the isolated DNA sequence. Small subregions or fragments of the nucleic acid encoding the spreading protein, for example 1-30 bases in length, can be prepared by standard, chemical synthesis. Antisense oligonucleotides and primers for use in the generation of larger synthetic fragment.

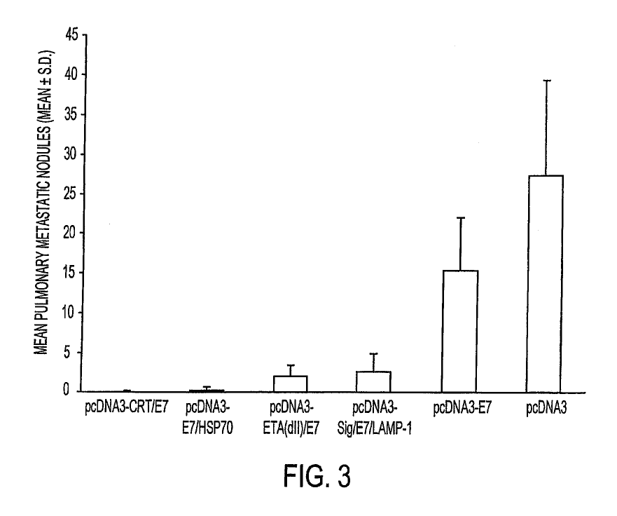

A preferred group of variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | |
|---|---|
| 1. Small aliphatic, nonpolar or slightly polar residues | Ala, Ser, Thr (Pro, Gly); |
| 2. Polar, negatively charged residues and their amides | Asp, Asn, Glu, Gln; |
| 3. Polar, positively charged residues | His, Arg, Lys; |
| 4. Large aliphatic, nonpolar residues | Met, Leu, Ile, Val (Cys) |
| 5. Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the wild-type or native protein in terms of its intercellular spreading activity and its ability to stimulate antigen specific T cell reactivity to an antigenic epitope or epitopes that are fused to the spreading protein. However, when it is difficult to predict the exact effect of the substitution, deletion or insertion in advance of doing so, one skilled in the art will appreciate that the effect can be evaluated by routine screening assays such as those described here, without requiring undue experimentation.

Whereas shorter chain variants can be made by chemical synthesis, for the present invention, the preferred longer chain variants are typically made by site-specific mutagenesis of the nucleic acid encoding the polypeptide, expression of the variant nucleic acid in cell culture, and, optionally, purification of the polypeptide from the cell culture, for example, by immunoaffinity chromatography using specific antibody immobilized to a column (to absorb the variant by binding to at least one epitope).

Exemplary fusion proteins provided herein comprise a calreticulin protein or homolog thereof and an antigen. For example, a fusion protein may comprise, consists essentially of, or consists of calreticulin or a calreticulin fragment, e.g., N-CRT, P-CRT and/or C-CRT, or an amino acid sequence that is at least about 90%, 95%, 96%, 97%, 98%, or 99% identical to the amino acid sequence of calreticulin, N-CRT, P-CRT or C-CRT, wherein the calreticulin fragment is functionally active as further described herein, linked to an antigen. A fusion protein may also comprise calreticulin or a calreticulin fragment and at least 1, 2, 3, 4, 5, 6, 7, 8, 9, 10 or more amino acids, or about 1-5, 1-10, 1-15, 1-20, 1-25, 1-30, 1-50 amino acids, at the N- and/or C-terminus of the calreticulin fragment. These additional amino acids may have an amino acid sequence that is unrelated to the amino acid sequence at the corresponding position in the calreticulin protein.

Homologs of calreticulin or calreticulin fragments may also comprise, consist essentially of, or consist of an amino acid sequence that differs from that of calreticulin, N-CRT, P-CRT or C-CRT by the addition, deletion, or substitution, e.g., conservative substitution, of at least about 1, 2, 3, 4, 5, 6, 7, 8, 9 or 10 amino acids, or from about 1-5, 1-10, 1-15 or 1-20 amino acids. Other homologs of calreticulin or calreticulin fragments are encoded by nucleotide sequences that are at least about 90%, 95%, 96%, 97%, 98%, or 99% identical to the nucleotide sequence encoding calreticulin, N-CRT, P-CRT or C-CRT, such as those described herein.

Yet other homologs of calreticulin or calreticulin fragments are encoded by nucleic acids that hybridize under stringent hybridization conditions to a nucleic acid that encodes calreticulin, N-CRT, P-CRT or C-CRT. For example, homologs may be encoded by nucleic acids that hybridize under high stringency conditions of 0.2 to 1×SSC at 65° C. followed by a wash at 0.2×SSC at 65° C. to a nucleic acid consisting of a sequence described herein. Nucleic acids that hybridize under low stringency conditions of 6×SSC at room temperature followed by a wash at 2×SSC at room temperature to nucleic acid consisting of a sequence described herein or a portion thereof can be used. Other hybridization conditions include 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

In the above composition the CRT or other stress protein preferably acts in potentiating an immune response by promoting: processing of the linked antigenic polypeptide via the MHC class I pathway or targeting of a cellular compartment that increases the processing. This basic strategy may be combined with an additional strategy pioneered by the present inventors and colleagues, that involve linking DNA encoding another protein, generically termed a “targeting polypeptide, to the antigen-encoding DNA. Again, for the sake of simplicity, the DNA encoding such a targeting polypeptide will be referred to herein as a “targeting DNA.” That strategy has been shown to be effective in enhancing the potency of the vectors carrying only antigen-encoding DNA. See for example, the following PCT publications by Wu et al: WO 01/29233; WO 02/009645; WO 02/061113; WO 02/074920; and WO 02/12281, all of which are incorporated by reference in their entirety. The other strategies include the use of DNA encoding polypeptides that promote or enhance:
(a) development, accumulation or activity of antigen presenting cells or targeting of antigen to compartments of the antigen presenting cells leading to enhanced antigen presentation;
(b) intercellular transport and spreading of the antigen; or
(c) any combination of (a) and (b).
(d) sorting of the lysosome-associated membrane protein type 1 (Sig/LAMP-1).
   The strategy includes use of:
(e) a viral intercellular spreading protein selected from the group of herpes simplex virus-1 VP22 protein, Marek's disease virus UL49 (see WO 02/09645), protein or a functional homologue or derivative thereof;
(f) other endoplasmic reticulum chaperone polypeptides selected from the group of CRT-like molecules ER60, GRP94, gp96, or a functional homologue or derivative thereof (see WO 02/12281, hereby incorporated by reference;
(g) a cytoplasmic translocation polypeptide domains of a pathogen toxin selected from the group of domain II of Pseudomonas exotoxin ETA or a functional homologue or derivative thereof;
(h) a polypeptide that targets the centrosome compartment of a cell selected from γ-tubulin or a functional homologue or derivative thereof; or
(i) a polypeptide that stimulates dendritic cell precursors or activates dendritic cell activity selected from the group of GM-CSF, Flt3-ligand extracellular domain, or a functional homologue or derivative thereof; or
(j) a costimulatory signal, such as a B7 family protein, including B7-DC (see U.S. Ser. No. 09/794,210), B7.1, B7.2, soluble CD40, etc.).
(k) an anti-apoptotic polypeptide preferably selected from the group consisting of (1) BCL-xL, (2) BCL2, (3) XIAP, (4) FLICEc-s, (5) dominant-negative caspase-8, (6) dominant negative caspase-9, (7) SPI-6, and (8) a functional homologue or derivative of any of (1)-(7). (See WO 2005/047501).

The details of the various targeting polypeptide strategies will not be discussed in detail herein. Of the IPP's above, the preferred alternatives to CRT are the other ER chaperone polypeptide exemplified by ER60, GRP94 or gp96, well-characterized ER chaperone polypeptide that representatives of the HSP90 family of stress-induced proteins (see WO 02/012281)

The experiments described herein demonstrate that the methods of the invention can enhance a cellular immune response, particularly, tumor-destructive CTL reactivity, induced by a DNA vaccine encoding an epitope of a human pathogen. Human HPV-16 E7 was used as a model antigen for vaccine development because human papillomaviruses (HPVs), particularly HPV-16, are associated with most human cervical cancers. The oncogenic HPV proteins E7 and E6 are important in the induction and maintenance of cellular transformation and co-expressed in most HPV-containing cervical cancers and their precursor lesions. Therefore, cancer vaccines, such as the compositions of the invention, that target E7 or E6 can be used to control of HPV-associated neoplasms (Wu, T-C, Curr Opin Immunol. 6:746-54, 1994).

However, as noted, the present invention is not limited to the exemplified antigen(s). Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Thus, in one embodiment, the antigen (e.g., the MHC class I-binding peptide epitope) is derived from a pathogen, e.g., it comprises a peptide expressed by a pathogen. The pathogen can be a virus, such as, e.g., a papilloma virus, a herpesvirus, a retrovirus (e.g., an immunodeficiency virus, such as HIV-1), an adenovirus, and the like. The papilloma virus can be a human papilloma virus; for example, the antigen (e.g., the Class I-binding peptide) can be derived from an HPV-16 E6 or E7 polypeptide. In a preferred embodiment, the HPV-16 E6 or E7 polypeptide used as an immunogen is substantially non-oncogenic, i.e., it does not bind retinoblastoma polypeptide (pRB) or binds pRB with such low affinity that the HPV-16 E7 polypeptide is effectively non-oncogenic when expressed or delivered in vivo, which is accomplished as described herein.

In alternative embodiments, the pathogen is a bacteria, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum.

In another embodiment, the MHC class I-binding antigenic peptide epitope is derived from a tumor cell. The tumor cell-derived peptide epitope can comprise a tumor associated antigen, e.g., a tumor specific antigen, such as, e.g., a HER-2/neu antigen, or one of a number of known melanoma antigens, etc.

In one embodiment, the isolated or recombinant nucleic acid molecule is operatively linked to a promoter, such as, e.g., a constitutive, an inducible or a tissue-specific promoter. The promoter can be expressed in any cell, including cells of the immune system, including, e.g., antigen presenting cells (APCs), e.g., in a constitutive, an inducible or a tissue-specific manner.

Unless defined otherwise, all technical and scientific terms used herein have the meaning commonly understood by a person skilled in the art of this invention. As used herein, the following terms have the meanings ascribed to them unless specified otherwise.

The term “antigen” or “immunogen” as used herein refers to a compound or composition comprising a peptide, polypeptide or protein which is “antigenic” or “immunogenic” when administered (or expressed in vivo by an administered nucleic acid, e.g., a DNA vaccine) in an appropriate amount (an “immunogenically effective amount”), i.e., capable of inducing, eliciting, augmenting or boosting a cellular and/or humoral immune response either alone or in combination or linked or fused to another substance (which can be administered at once or over several intervals). An immunogenic composition can comprise an antigenic peptide of at least about 5 amino acids, a peptide of 10 amino acids in length, a polypeptide fragment of 15 amino acids in length, 20 amino acids in length or longer. Smaller immunogens may require presence of a “carrier” polypeptide e.g., as a fusion protein, aggregate, conjugate or mixture, preferably linked (chemically or otherwise) to the immunogen. The immunogen can be recombinantly expressed from a vaccine vector, which can be naked DNA comprising the immunogen's coding sequence operably linked to a promoter, e.g., an expression cassette as described herein. The immunogen includes one or more antigenic determinants or epitopes which may vary in size from about 3 to about 15 amino acids.

The term “vaccine” is used interchangeably with “immunogen” when referring to the DNA compositions of the present invention. Similarly, the terms “vaccinate” and “immunize” are used interchangeably here.

The term “epitope” as used herein refers to an antigenic determinant or antigenic site that interacts with an antibody or a T cell receptor (TCR), e.g., the MHC class I-binding peptide compositions (or expressed products of the nucleic acid compositions of the invention) used in the methods of the invention. An “antigen” is a molecule or chemical structure that either induces an immune response or is specifically recognized or bound by the product or mediator of an immune response, such as an antibody or a CTL. The specific conformational or stereochemical “domain” to which an antibody or a TCR bind is an “antigenic determinant” or “epitope.” TCRs bind to peptide epitopes which are physically associated with a third molecule, a major histocompatibility complex (MHC) class I or class II protein.

The term “recombinant” refers to (1) a nucleic acid or polynucleotide synthesized or otherwise manipulated in vitro, (2) methods of using recombinant DNA technology to produce gene products in cells or other biological systems, or (3) a polypeptide encoded by a recombinant nucleic acid. For example, the CRT-encoding nucleic acid or polypeptide, the nucleic acid encoding an MHC class I-binding peptide epitope (antigen) or the peptide itself is preferably be recombinant. “Recombinant means” includes ligation of nucleic acids having various coding regions or domains or promoter sequences from different sources into a single unit in the form of an expression cassette or vector for expression of the coding sequences in the vectors resulting in production of the encoded polypeptide.

General Recombinant DNA Methods

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, D M, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation of nucleic acids, such as, e.g., generating mutations in sequences, subcloning, labeling probes, sequencing, hybridization and the like are well described in the scientific and patent literature. See, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Amplification of Nucleic Acids

Oligonucleotide primers can be used to amplify nucleic acids to generate fusion protein coding sequences used to practice the invention, to monitor levels of vaccine after in vivo administration (e.g., levels of a plasmid or virus), to confirm the presence and phenotype of activated CTLs, and the like. The skilled artisan can select and design suitable oligonucleotide amplification primers using known sequences. Amplification methods are also well known in the art, and include, e.g., polymerase chain reaction, PCR (PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (Wu (1989) Genomics 4:560; Landegren (1988) Science 241:1077; Barringer (1990) Gene 89:117); transcription amplification (Kwoh (1989) Proc. Natl. Acad. Sci. USA 86:1173); and, self-sustained sequence replication (Guatelli (1990) Proc. Natl. Acad. Sci. USA 87:1874); Qβ replicase amplification (Smith (1997) J. Clin. Microbiol. 35:1477-1491; Burg (1996) Mol. Cell. Probes 10:257-271) and other RNA polymerase mediated techniques (NASBA, Cangene, Mississauga, Ontario; Berger (1987) Methods Enzymol. 152:307-316; U.S. Pat. Nos. 4,683,195 and 4,683,202; Sooknanan (1995) Biotechnology 13:563-564).

Unless otherwise indicated, a particular nucleic acid sequence is intended to encompasses conservative substitution variants thereof (e.g., degenerate codon substitutions) and a complementary sequence. The term “nucleic acid” is synonymous with “polynucleotide” and is intended to include a gene, a cDNA molecule, an mRNA molecule, as well as a fragment of any of these such as an oligonucleotide, and further, equivalents thereof (explained more fully below). Sizes of nucleic acids are stated either as kilobases (kb) or base pairs (bp). These are estimates derived from agarose or polyacrylamide gel electrophoresis (PAGE), from nucleic acid sequences which are determined by the user or published. Protein size is stated as molecular mass in kilodaltons (kDa) or as length (number of amino acid residues). Protein size is estimated from PAGE, from sequencing, from presumptive amino acid sequences based on the coding nucleic acid sequence or from published amino acid sequences.

Specifically, cDNA molecules encoding the amino acid sequence corresponding to the fusion polypeptide of the present invention or fragments or derivatives thereof can be synthesized by the polymerase chain reaction (PCR) (see, for example, U.S. Pat. No. 4,683,202) using primers derived the sequence of the protein disclosed herein. These cDNA sequences can then be assembled into a eukaryotic or prokaryotic expression vector and the resulting vector can be used to direct the synthesis of the fusion polypeptide or its fragment or derivative by appropriate host cells, for example COS or CHO cells.

This invention includes isolated nucleic acids having a nucleotide sequence encoding the novel fusion polypeptides that comprise a translocation polypeptide and an antigen, fragments thereof or equivalents thereof. The term nucleic acid as used herein is intended to include such fragments or equivalents. The nucleic acid sequences of this invention can be DNA or RNA.

A cDNA nucleotide sequence the fusion polypeptide can be obtained by isolating total mRNA from an appropriate cell line. Double stranded cDNA is prepared from total mRNA. cDNA can be inserted into a suitable plasmid, bacteriophage or viral vector using any one of a number of known techniques.

In reference to a nucleotide sequence, the term “equivalent” is intended to include sequences encoding structurally homologous and/or a functionally equivalent proteins. For example, a natural polymorphism in a nucleotide sequence encoding an anti-apoptotic polypeptide according to the present invention (especially at the third base of a codon) may be manifest as “silent” mutations which do not change the amino acid sequence. Furthermore, there may be one or more naturally occurring isoforms or related, immunologically cross-reactive family members of these proteins. Such isoforms or family members are defined as proteins that share function amino acid sequence similarity to the reference polypeptide.

Fragment of Nucleic Acid

A fragment of a nucleic acid sequence is defined as a nucleotide sequence having fewer nucleotides than the nucleotide sequence encoding the full length CRT polypeptide, antigenic polypeptide, or the fusion thereof. This invention includes such nucleic acid fragments that encode polypeptides which retain (1) the ability of the fusion polypeptide to induce increases in frequency or reactivity of T cells, preferably CD8+ T cells, that are specific for the antigen part of the fusion polypeptide.

For example, a nucleic acid fragment as intended herein encodes an antigen, or encodes CRT or a homologue, domain, or fragment thereof, that retains the ability to improve the immunogenicity of an antigen-only DNA vaccine when administered as a chimeric DNA with antigen-encoding sequence.

Generally, the nucleic acid sequence encoding a fragment of an anti-apoptotic polypeptide comprises of nucleotides from the sequence encoding the mature protein (or an active fragment thereof).

Nucleic acid sequences of this invention may also include linker sequences, natural or modified restriction endonuclease sites and other sequences that are useful for manipulations related to cloning, expression or purification of encoded protein or fragments. These and other modifications of nucleic acid sequences are described herein or are well-known in the art.

The techniques for assembling and expressing DNA coding sequences for translocation types of proteins, and DNA coding sequences for antigenic polypeptides, include synthesis of oligonucleotides, PCR, transforming cells, constructing vectors, expression systems, and the like; these are well-established in the art such that those of ordinary skill are familiar with standard resource materials, specific conditions and procedures.

Expression Vectors and Host Cells

This invention includes an expression vector comprising a nucleic acid sequence encoding (a) an antigen linked to (b) an IPP and operably linked to at least one regulatory sequence, which includes a promoter that is expressable in a eukaryotic cell, preferably in a mammalian cells, more preferably in a human cell.

The term “expression vector” or “expression cassette” as used herein refers to a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879), and includes both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Those skilled in the art appreciate that the particular design of an expression vector of this invention depends on considerations such as the host cell to be transfected and/or the type of protein to be expressed.

The present expression vectors comprise the full range of nucleic acid molecules encoding the various embodiments of the fusion polypeptide and its functional derivatives (defined herein) including polypeptide fragments, variants, etc.

Such expression vectors may be used to transfect host cells (in vitro, ex vivo or in vivo) for expression of the DNA and production of the encoded proteins which include fusion proteins or peptides. It will be understood that a genetically modified cell expressing the fusion polypeptide may transiently express the exogenous DNA for a time sufficient for the cell to be useful for its stated purpose.

The present in invention provides methods for producing the fusion polypeptides, fragments and derivatives. For example, a host cell transfected with a nucleic acid vector that encodes the fusion polypeptide is cultured under appropriate conditions to allow expression of the polypeptide.

Host cells may also be transfected with one or more expression vectors that singly or in combination comprise (a) DNA encoding at least a portion of the fusion polypeptide and (b) DNA encoding at least a portion of a second protein, preferably an antigen, so that the host cells produce yet further fusion polypeptides.

A culture typically includes host cells, appropriate growth media and other byproducts. Suitable culture media are well known in the art. The fusion polypeptide can be isolated from medium or cell lysates using conventional techniques for purifying proteins and peptides, including ammonium sulfate precipitation, fractionation column chromatography (e.g. ion exchange, gel filtration, affinity chromatography, etc.) and/or electrophoresis (see generally, “Enzyme Purification and Related Techniques”, Meth Enzymol, 22:233-577 (1971)). Once purified, partially or to homogeneity, the recombinant polypeptides of the invention can be utilized in pharmaceutical compositions as described in more detail herein.

The term “isolated” as used herein, when referring to a molecule or composition, such as a translocation polypeptide or a nucleic acid coding therefor, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transformed or transfected to express the fusion polypeptide or a homologue or functional derivative thereof are within the scope of the invention. For example, the fusion polypeptide may be expressed in yeast, or mammalian cells such as Chinese hamster ovary cells (CHO) or, preferably human cells. Preferred cells for expression according to the present invention are APCs most preferably, DCs. Other suitable host cells are known to those skilled in the art.

Expression in eukaryotic cells leads to partial or complete glycosylation and/or formation of relevant inter- or intra-chain disulfide bonds of the recombinant protein.

Often, in fusion expression vectors, a nucleotide sequence encoding a proteolytic cleavage site is introduced at the junction of the reporter group and the target protein to enable separation of the target protein from the reporter group subsequent to purification of the fusion protein. Proteolytic enzymes for such cleavage and their recognition sequences include Factor Xa, thrombin and enterokinase. Typical fusion expression vectors include pGEX (Amrad Corp., Melbourne, Australia), pMAL (New England Biolabs) and pRIT5 (Pharmacia, Piscataway, N.J.) which fuse glutathione S-transferase, maltose E binding protein, or protein A, respectively, to the target recombinant protein. Inducible non-fusion expression vectors include pTrc (Amann et al., Gene 69:301-15, 1988) and pET 11d (Studier et al., Gene Expression Technology Meth Enzymol 185:60-89, Academic Press, 1990).

Vector Construction

Construction of suitable vectors comprising the desired coding and control sequences employs standard ligation and restriction techniques which are well understood in the art. Isolated plasmids, DNA sequences, or synthesized oligonucleotides are cleaved, tailored, and re-ligated in the form desired. The sequences of several preferred plasmid vectors, with and without inserted coding sequences, have been disclosed above.

The DNA sequences which form the vectors are available from a number of sources. Backbone vectors and control systems are generally found on available “host” vectors which are used for the bulk of the sequences in construction. For the pertinent coding sequence, initial construction may be, and usually is, a matter of retrieving the appropriate sequences from cDNA or genomic DNA libraries. However, once the sequence is disclosed it is possible to synthesize the entire gene sequence in vitro starting from the individual nucleotide derivatives. The entire gene sequence for genes of sizeable length, e.g., 500-1000 bp may be prepared by synthesizing individual overlapping complementary oligonucleotides and filling in single stranded nonoverlapping portions using DNA polymerase in the presence of the deoxyribonucleotide triphosphates. This approach has been used successfully in the construction of several genes of known sequence.

Synthetic oligonucleotides are prepared by either the phosphotriester method as described by references cited above or the phosphoramidite method (Beaucage, S L et al., Tet Lett 22:1859, 1981; Matteucci, M D et al., J Am Chem Soc 103:3185, 1981) and can be prepared using commercially available automated oligonucleotide synthesizers. Kinase treatment of single strands prior to annealing or for labeling is by conventional methods.

Once the components of the desired vectors are thus available, they can be excised and ligated using standard restriction and ligation procedures. Site-specific DNA cleavage is performed by treating with the suitable restriction enzyme (or enzymes) under conditions which are understood in the art, and the particulars of which are specified by the manufacturer of these commercially available restriction enzymes. See, e.g., New England Biolabs, Product Catalog; Meth Enzymol. 65:499-560, 1980.

Restriction cleaved fragments may be blunt ended by treating with the large fragment of E. coli DNA polymerase I (Klenow) in the presence of the four deoxynucleotide triphosphates (dNTPs) using conventional methods and conditions. Ligations are performed using conventional methods. In vector construction employing “vector fragments”, the fragment is commonly treated with bacterial or mammalian alkaline phosphatase to remove the 5′ phosphate and prevent self-ligation. Alternatively, re-ligation can be prevented in vectors which have been double digested by additional restriction enzyme and separation of the unwanted fragments.

Any of a number of methods are used to introduce mutations into the coding sequence to generate the variants of the invention. These mutations include simple deletions or insertions, systematic deletions, insertions or substitutions of clusters of bases or substitutions of single bases.

For example, modifications of DNA sequences are created by site-directed mutagenesis, a well-known technique for which protocols and reagents are commercially available (Zoller, M J et al., Nucleic Acids Res 10:6487-500, 1982; Adelman, J P et al., DNA 2:183-193, 1983). Using conventional methods, transformants are selected based on the presence of a selectable marker such as an antibiotic resistance gene depending on the mode of plasmid construction.

Vector DNA can be introduced into mammalian cells via conventional techniques such as calcium phosphate or calcium chloride co-precipitation, DEAE-dextran-mediated transfection, lipofection, or electroporation. Suitable methods for transforming host cells can be found in Sambrook et al. supra and other standard texts. In fusion expression vectors, a proteolytic cleavage site may be introduced at the junction of two sequences (such as a reporter group and the target protein to enable separation of the target protein from the reporter group subsequent to purification of the fusion protein). Proteolytic enzymes for such cleavage and their recognition sequences include Factor Xa, thrombin and enterokinase.

Promoters and Enhancers

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

The preferred promoter sequences of the present invention must be operable in mammalian cells and may be either eukaryotic or viral promoters. Although preferred promoters are described in the Examples, other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other preferred viral promoters include that present in the CMV-LTR (from cytomegalovirus) (Bashart, M. et al., Cell 41:521, 1985) or in the RSV-LTR (from Rous sarcoma virus) (Gorman, C. M., Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, P A, et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993.

The promoter region may further include an octamer region which may also function as a tissue specific enhancer, by interacting with certain proteins found in the specific tissue. The enhancer domain of the DNA construct of the present invention is one which is specific for the target cells to be transfected, or is highly activated by cellular factors of such target cells. Examples of vectors (plasmid or retrovirus) are disclosed in (Roy-Burman et al., U.S. Pat. No. 5,112,767). For a general discussion of enhancers and their actions in transcription, see, Lewin, B M, Genes IV, Oxford University Press pp. 552-576, 1990 (or later edition). Particularly useful are retroviral enhancers (e.g., viral LTR) that is preferably placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Nucleic acids of the invention can also be chemically synthesized using standard techniques, including solid-phase synthesis which, like peptide synthesis, has been fully automated with commercially available DNA synthesizers (Itakura U.S. Pats. No. 4,598,049, 4,401,796 and 4,373,071; Caruthers et al. U.S. Pat. No. 4,458,066.

Therapeutic Compositions and their Administration

A vaccine composition comprising the nucleic acid encoding the antigen in a fusion polypeptide with an IPP, here CRT or a homologue or derivative thereof, a particle comprising the nucleic acid or a cell expressing this nucleic acid, is administered to a mammalian subject. The vaccine composition is administered in a pharmaceutically acceptable carrier in a biologically-effective or a therapeutically-effective amount.

Certain preferred conditions are disclosed in the Examples. The composition may be given alone or in combination with another protein or peptide such as an immunostimulatory molecule. Treatment may include administration of an adjuvant, used in its broadest sense to include any nonspecific immune stimulating compound such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents.

Thus an effective amount of the vaccine is between about 1 nanogram and about 1 gram per kilogram of body weight of the recipient, more preferably between about 0.1 μg/kg and about 10 mg/kg, more preferably between about 1 μg/kg and about 1 mg/kg. Dosage forms suitable for internal administration preferably contain (for the latter dose range) from about 0.1 μg to 100 μg of active ingredient per unit. The active ingredient may vary from 0.5 to 95% by weight based on the total weight of the composition. Alternatively, an effective dose of cells transfected with the DNA vaccine constructs of the present invention is between about 10⁴ and 10⁸ cells. Those skilled in the art of immunotherapy will be able to adjust these doses without undue experimentation.

The composition may be administered in a convenient manner, e.g., injection by a convenient and effective route.

Preferred routes for the DNA include (a) intradermal “gene gun” delivery wherein DNA-coated gold particles in an effective amount are delivered using a helium-driven gene gun (BioRad, Hercules, Calif.) with a discharge pressure set at a known level, e.g., of 400 p.s.i.; (b) i.m. injection using a conventional syringe needle; and (c) use of a needle-free biojector such as the Biojector 2000 (Bioject Inc., Portland, Oreg.) which is an injection device consisting of an injector and a disposable syringe. The orifice size controls the depth of penetration. For example, 50 μg of DNA may be delivered using the Biojector with no. 2 syringe nozzle.

Depending on the route of administration, the composition may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol). or in an appropriate carrier such as liposomes (including water-in-oil-in-water emulsions as well as conventional liposomes (Strejan et al., J. Neuroimmunol 7:27, 1984).

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions are preferably formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Other pharmaceutically acceptable carriers for the nucleic acid vaccine compositions according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

Delivery of Vaccine Nucleic Acid to Cells and Animals

The following references set forth principles and current information in the field of basic, medical and veterinary virology and are incorporated by reference: Fields Virology, Fields, B N et al., eds., Lippincott Williams & Wilkins, NY, 1996; Principles of Virology: Molecular Biology, Pathogenesis, and Control, Flint, S. J. et al., eds., Amer Soc Microbiol, Washington D.C., 1999; Principles and Practice of Clinical Virology, 4th Edition, Zuckerman. A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy Classification and Nomenclature of Viruses: Seventh Report of the International Committee on Taxonomy of Viruses, by M. H. V. Van Regenmortel, M H V et al., eds., Academic Press; NY, 2000.

The Examples below describe certain preferred approaches to delivery of the vaccines and combinations of the present invention, intradermally by gene gun or intramuscularly. A broader description of other approaches including viral and nonviral vectors and delivery mechanisms follow.

DNA delivery involves introduction of a “foreign” DNA into a cell ex vivo and ultimately, into a live animal or directly into the animal. Several general strategies for gene delivery (=delivery of nucleic acid vectors) for purposes that include “gene therapy” have been studied and reviewed extensively (Yang, N-S., Crit. Rev. Biotechnol. 12:335-356 (1992); Anderson, W F, Science 256:808-13, 1992; Miller, A S, Nature 357:455-60, 1992; Crystal, R G, Amer. J. Med. 92(suppl 6A):44-52S, 1992; Zwiebel, J A et al., Ann NY Acad Sc. 618:394-404, 1991; McLachlin, J R et al., Prog. Nucl. Acid Res. Molec. Biol. 38:91-135, 1990; Kohn, D B et al., Cancer Invest. 7:179-92, 1989), which references are herein incorporated by reference in their entirety).

One approach comprises nucleic acid transfer into primary cells in culture followed by autologous transplantation of the ex vivo transformed cells into the host, either systemically or into a particular organ or tissue.

The term “systemic administration” refers to administration of a composition or agent such as a DNA vaccine as described herein, in a manner that results in the introduction of the composition into the subject's circulatory system or otherwise permits its spread throughout the body.

“Regional” administration refers to administration into a specific, and somewhat more limited, anatomical space, such as intraperitoneal, intrathecal, subdural, or to a specific organ.

“Local administration” refers to administration of a composition or drug into a limited, or circumscribed, anatomic space, such as intratumoral injection into a tumor mass, subcutaneous injections, intradermal or intramuscular injections

Those of skill in the art will understand that local administration or regional administration may also result in entry of a composition into the circulatory system—i.e., rendering it systemic to one degree or another.

For accomplishing the objectives of the present invention, nucleic acid therapy would be accomplished by direct transfer of a the functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the antigen-containing expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

The DNA molecules encoding the fusion polypeptides of the present invention may also be packaged into retrovirus vectors using packaging cell lines that produce replication-defective retroviruses, as is well-known in the art (e.g., Cone, R. D. et al., Proc Natl Acad Sci USA 81:6349-53, 1984; Mann, R F et al., Cell 33:153-9, 1983; Miller, A D et al., Molec Cell Biol 5:431-7, 1985; Sorge, J, et al., Molec Cell Biol 4:1730-7, 1984; Hock, R A et al., Nature 320:257, 1986; Miller, A D et al., Molec Cell Biol 6:2895-2902 (1986). Newer packaging cell lines which are efficient an safe for gene transfer have also been described (Bank et al., U.S. Pat. No. 5,278,056).

The above approach can be utilized in a site specific manner to deliver the retroviral vector to the tissue or organ of choice. Thus, for example, a catheter delivery system can be used (Nabel, E G et al., Science 244:1342 (1989)). Such methods, using either a retroviral vector or a liposome vector, are particularly useful to deliver the nucleic acid to be expressed to a blood vessel wall, or into the blood circulation of a tumor.

Other virus vectors may also be used, including recombinant adenoviruses (Horowitz, M S, In: Virology, Fields, B N et al., eds, Raven Press, NY, 1990, p. 1679; Berkner, K L, Biotechniques 6:616-29, 1988; Strauss, S E, In: The Adenoviruses, Ginsberg, H S, ed., Plenum Press, NY, 1984, chapter 11), herpes simplex virus (HSV) for neuron-specific delivery and persistence. Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941, 1991) according to the present invention.

Another vector which can express the DNA molecule of the present invention, and is useful in the present therapeutic setting is vaccinia virus, which can be rendered non-replicating (U.S. Pat. Nos. 5,225,336; 5,204,243; 5,155,020; 4,769,330; Fuerst, T R et al., Proc. Natl. Acad. Sci. USA 86:2549-53, 1992; Chakrabarti, S et al., Mol Cell Biol 5:3403-9, 1985). Descriptions of recombinant vaccinia viruses and other viruses containing heterologous DNA and their uses in immunization and DNA therapy are reviewed in: Moss, B, Curr Opin Genet Dev 3:86-90, 1993; Moss, B, Biotechnol. 20:345-62, 1992);.

In addition to naked DNA or viral vectors, engineered bacteria may be used as vectors. A number of bacterial strains including Salmonella, BCG and Listeria monocytogenes (LM) (Hoiseth et al., Nature 291:238-9, 1981; Poirier, T P et al., J Exp Med 168:25-32, 1988); Sadoff, J C et al., Science 240:336-8, 1988; Stover, C K et al., Nature 351:456-60, 1991; Aldovini, A et al., Nature 351:479-82, 1991). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA (Wolff et al., 1990, supra) and particle-bombardment mediated gene transfer (Yang, N-S, et al., Proc Natl Acad Sci USA 87:9568, 1990; Williams, R S et al., Proc Natl Acad Sci USA 88:2726, 1991; Zelenin, A V et al., FEBS Lett 280:94, 1991; Zelenin, A V et al., FEBS Lett 244:65, 1989); Johnston, S A et al., In Vitro Cell Dev Biol 27:11, 1991). Furthermore, electroporation, a well-known means to transfer genes into cell in vitro, can be used to transfer DNA molecules according to the present invention to tissues in vivo (Titomirov, A V et al., Biochim Biophys Acta 1088:131, 1991).

“Carrier mediated gene transfer” has also been described (Wu, C H et al., J Biol Chem 264:16985, 1989; Wu, G Y et al., J Biol Chem 263:14621, 1988; Soriano, P et al., Proc Nat. Acad Sci USA 80:7128, 1983; Wang, C-Y et al., Pro. Natl Acad Sci USA 84:7851, 1982; Wilson, J M et al., J Biol Chem 267:963, 1992). Preferred carriers are targeted liposomes (Nicolau, C et al., Proc Natl Acad Sci USA 80:1068, 1983; Soriano et al., supra) such as immunoliposomes, which can incorporate acylated mAbs into the lipid bilayer (Wang et al., supra). Polycations such as asialoglycoprotein/polylysine (Wu et al., 1989, supra) may be used, where the conjugate includes a target tissue-recognizing molecule (e.g., asialo-orosomucoid for liver) and a DNA binding compound to bind to the DNA to be transfected without causing damage, such as polylysine. This conjugate is then complexed with plasmid DNA of the present invention.

Plasmid DNA used for transfection or microinjection may be prepared using methods well-known in the art, for example using the Quiagen procedure (Quiagen), followed by DNA purification using known methods, such as the methods exemplified herein.

### Example 1

### Materials and Methods

Plasmid DNA Construction

The generation of pcDNA3, pcDNA3-E7, pcDNA3-CRT/E7 (Cheng et al., supra), pcDNA3-E7/HSP70 (Chen C H et al., Cancer Res 60:1035-42, 2000), and pcDNA3-ETA(dII)/E7 (Hung C F et al., Cancer Res 2001; 61:3698-3703) has been described previously. To generate pcDNA3-Sig/E7/LAMP-1, Sig/E7/LAMP-1 was cut at the EcoRI/BamHI sites from pCMV(neo)-Sig/E7/LAMP-1 (Ji H et al., Hum Gene Ther 10:2727-40, 1999) and cloned into pcDNA3.

For generation of pNGVL4a-E7(detox), the E7 gene was cloned into pNGVL4a (National Gene Vector Laboratory) using the EcoRI and KpnI restriction sites. Using site-directed mutagenesis, two point mutations, which had previously been found to reduce Rb binding (Munger K et al., EMBO J 8:4099-4105, 1989), were introduced into the E7 gene. The primers used to introduce these mutations were as follows:

E7(detox) Forward:

(SEQ ID NO:21)

5′ ctgatctctacggttatgggcaattaaatgacagctc 3′
and
E7(detox) Reverse:

(SEQ ID NO:22)

5′ gagctgtcatttaattgcccataaccgtagagatca 3′.
For generation of pNGVL4a-CRT/E7(detox), CRT was
PCR amplified with the following primers

(SEQ ID NO:23)

5′ aaagtcgacatgctgctatccgtgccgctgc 3′
and

(SEQ ID NO:24)

5′ gaattcgttgtctggccgcacaatca 3′.

using a human CRT plasmid as a template (which was kindly provided by Dr. David Llewellyn of Dept. Medical Biochemistry at University of Wales College of Medicine at Cardiff, U.K.). The PCR product was cut with Sal I/EcoRI and cloned into the Sal I/EcoRI sites of pNGVL4a-E7(detox). The accuracy of DNA constructs was confirmed by DNA sequencing.

For the generation of pcDNA3-N-CRT, DNA encoding the N-domain of CRT, N-CRT was first amplified with PCR by using rabbit CRT cDNA as the template (Michalak, M., et al., Biochem Cell Biol 76:779-85, 1998) and a set of primers:

[SEQ ID NO:25]

5′-ccggtctagaacgctgctccctgtgccgct-3′
and

[SEQ ID NO:26]

5′-cccgaattcgttgtccgggccgcacgatca-3′.

The amplified product was further cloned into the XbaI/EcoRI site of pcDNA3 (Invitrogen Corp)

For the generation of pcDNA3-P-CRT, DNA encoding the P-domain of CRT was first amplified with PCR using rabbit CRT cDNA as the template and a set of primers,

[SEQ ID NO:27]

5′-tgctctagaatgtacaagggtgagtggaagcc-3′
and

[SEQ ID NO:28]

5′-ccggaattccagctcgtccttggcctggcc-3′.

The amplified product was further cloned into the XbaI/EcoRI site of pcDNA3.

For the generation of pcDNA3-C-CRT, DNA encoding the C-domain of CRT was first amplified with PCR using rabbit CRT cDNA as the template and a set of primers,

[SEQ ID NO:29]

5′-tgctctagaatgtacaagggtgagtggaagc-3′
and

[SEQ ID NO:30]

5′-ccggaattccagctcgtccttggcctggc-3′..

The completed product was then cloned into the XbaI/EcoRI site of pcDNA3.

For the generation of pcDNA3-N-CRT/E7, P-CRT/E7, and C-CRT/E7, DNA encoding E7 was first amplified with pcDNA3-E7 as a template and a set of primers,

[SEQ ID NO:31]

5′-ggggaattcatggagatacaccta-3′
and

[SEQ ID NO:32]

5′-ggtggatccttgagaacagatgg-3′,

and then cloned into the EcoRI/BamHI sites of pcDNA3-N-CRT, pcDNA3-P-CRT, or pcDNA3-C-CRT to generate pcDNA3-N-CRT/E7, pcDNA3-P-CRT/E7, or pcDNA3-C-CRT/E7.
Mice

Six- to eight-week-old female C57BL/6 mice were purchased from the National Cancer Institute (Frederick, Md.) and kept in the oncology animal facility of the Johns Hopkins Hospital (Baltimore, Md.). IFN-γ knockout mice were obtained from the Jackson Laboratory (Bar Harbor, Me.). All animal procedures were performed according to approved protocols and in accordance with the recommendations for the proper use and care of laboratory animals.

Generation of TC-1 and TC-1 P3 (A15) Tumor Cell Line

The production and maintenance of TC-1 cells has been described by Lin et al. supra.

For the generation of TC-1 P3 (A15), Vac-Sig/E7/LAMP-1-vaccinated mice were challenged with TC-1 tumor cells. Vaccination with Vac-Sig/E7/LAMP-1 elicits E7-specific antitumor responses against HPV-16 E7-expressing tumors (TC-1), although the vaccine fails to prevent tumor formation in approximately 20% of the vaccinated mice (Lin et al., supra). The outgrown TC-1 tumors from Vac-Sig/E7/LAMP-1 vaccinated mice were explanted, cut into pieces of less than 1 mm in diameter, digested with collagenase at a concentration of 1 mg/mil in DMEM (GIBCO BRL, Rockville, Md.), and expanded in vitro. These expanded cell lines were called TC-1 P1. Vac-Sig/E7/LAMP-1 vaccinated mice were then challenged with TC-1 P1 tumor cells. Approximately 40% of vaccinated mice developed tumors (not shown). The outgrown tumors from these vaccinated mice were then explanted and expanded in vitro to create the TC-1 P2 tumor cell line. Vac-Sig/E7/LAMP-1 vaccinated mice were then challenged with TC-1 P2 tumor cells. This time approximately 60-80% of vaccinated mice developed tumors (not shown). The outgrown tumors from these vaccinated mice were further explanted and expanded in vitro to generate the TC-1 P3 tumor cell line. 50 TC-1 P3 clones were generated by limiting dilution. Among the TC-1 P3 clones, a representative clone with marked down-regulation of MHC class I expression was isolated and expanded, creating the TC-1 P3 (A15) tumor cell line. We have determined that more than 90% of the TC-1 P3 (A15) cells exhibited down-regulated MHC class I expression. Less than 10% of the TC-1 P3 (A15) cells expressed any MHC class 1 molecules. Monoclonal antibody (mAb) Anti-H-2K^{b}/H-2D^{b} (clone 28-8-6) (BD Bioscience, San Diego, Calif.) was used to detect MHC class I expression. Both TC-1 and TC-1 P3 (A15) cells were grown in RPMI 1640, supplemented with 10% (v/v) fetal bovine serum, 50 units/ml penicillin/streptomycin, 2 mM L-glutamine, 1 mM sodium pyruvate, 2 mM nonessential amino acids, and 0.4 mg/ml G418 at 37° C. with 5% CO₂. On the day of tumor challenge, tumor cells were harvested by trypsinization, washed twice with 1× Hanks buffered salt solution (HBSS), and resuspended in 1×HBSS to the designated concentration for injection.

DNA Vaccination

For the gene gun mediated intradermal vaccination, DNA-coated gold particles (1 μg DNA/bullet) were delivered to the shaved abdominal region of C57BL/6 mice using a helium-driven gene gun (BioRad, Hercules, Calif.) with a discharge pressure of 400 p.s.i., as previously described (Chen C H et al. Cancer Res 60: 1035-42, 2000). C57BL/6 Mice were vaccinated via gene gun with either 2 μg of pcDNA3, pcDNA3-E7, pcDNA3-CRT/E7, pcDNA3-E7/HSP70, pcDNA3-ETA(dII)/E7, pcDNA3-Sig/E7/LAMP-1, pNGVL4a, pNGVL4a-E7(detox), or pNGVL4a-CRT/E7(detox). These mice received a booster with the same regimen one week later.

For the intramuscular (i.m.) DNA vaccination, 50 μg/mouse of pNGVL4a, pNGVL4a-E7(detox), and pNGVL4a-CRT/E7(detox) DNA vaccines were delivered i.m. by syringe needle injection. These mice received a booster with the same regimen one week later.

Intracellular Cytokine Staining and Flow Cytometry Analysis

Staining of cell surface CD8 and intracellular IFN-γ as well as FACScan analysis was performed as described previously (Chen et al., supra) Prior to FACScan, spleen cells from different groups of mice were collected and incubated for 20 hours with 1 μg/mil of E7 peptide (aa 49-57, RAHYNIVTF, SEQ ID NO:33) (Feltkamp M C et al., Eur J Immunol 23:2242-49, 1993) containing an MHC class I epitope for detecting E7-specific CD8+ T cell precursors. Golgistop (Brefeldin A) was added 6 hours before harvesting the cells from the culture. Triplicate experiments were performed using a pool of spleen cells from the group of mice vaccinated with the same vaccine construct. The number of IFN-γ-secreting CD8⁺ T cells was analyzed using flow cytometry. Analysis was performed on a Becton-Dickinson FACScan with CELLQuest software (Becton Dickinson Immunocytometry System, Mountain View, Calif.).

In Vivo Tumor Therapy (Using TC-1 Tumors)

For in vivo tumor therapy experiments using an E7-expressing tumor (TC-1), mice (5/group) were challenged intravenously (iv) through the tail vein with 10⁴ TC-1 cells. Seven days after tumor challenge, mice were administered 2 μg of various DNA vaccines or control plasmids via gene gun. One week after the first vaccination, the mice were boosted with the same regimen. Mice were sacrificed and lungs explanted on day 28. The pulmonary nodules on the surface of the lungs of each mouse were counted by experimenters blinded to sample identity as described previously (Ji et al., supra).

Long Term (8 Week) In Vivo Tumor Protection Experiment

Mice (5/group) were vaccinated via gene gun with 2 μg of various DNA vaccines or control plasmids. One week later, mice were boosted with the same regimen. Eight weeks after the initial vaccination, mice were challenged iv with 10⁵ TC-1 cells via tail vein. Mice were sacrificed 28 days after the tumor challenge and lung surface pulmonary nodules in each mouse were counted by experimenters blinded to sample identity.

In Vivo Tumor Protection Against TC-1 P3 (A15) Class I Down-Regulated Tumors.

C57BL/6 mice (5/group) were vaccinated with various DNA vaccines or control plasmids as described below by gene gun injection. One week after the last vaccination, mice were challenged with 5×10⁴ TC-1 P3 (A15) tumor cells by subcutaneous (s.c.) injection in the right leg. Tumor growth was monitored by visual inspection and palpation twice weekly as described previously (Lin et al., supra).

For studies comparing wild type and IFN-γ KO C57BL/6 mice and mice, mice (5/group) were vaccinated via gene gun with 2 μg of pcDNA3-CRT/E7 DNA and then boosted with the same regimen one week later. One week after the last vaccination, mice were challenged with 5×10⁴ TC-1 P3 (A15) tumor cells sc in the right leg. Tumor growth was monitored by visual inspection and palpation twice weekly.

Immunohistochemical Labeling for the Quantitation of Microvessel Density

Labeling of intratumoral microvessels was performed with rat anti-mouse CD31 mAb (1:30 dilution, Bioscience), followed by VECTOR® M.O.M Immunodetection Kit (VECTOR; Burlingame, Calif.) using a known method (Cheng, W F et al., Cancer 85:651-7, 1999). In each section, the three most vascularized areas were selected and microvessels counted at 200× magnification; the mean MVD count of the three fields for each tumor was calculated count. Large vessels with thick muscular walls and lumina with diameters accommodating >˜8 blood cells were excluded. MVDs were evaluated in tumors of similar size. All measurements were performed by a single pathologist, blinded to sample identity.

In Vivo Angiogenesis Assay Using Matrigel®

In vivo angiogenesis was assessed using the Matrigel® plug assay, basically according to Cheng et al., 2001, supra and Coughlin, C M et al., J Clin Invest 101: 1441-52, 1998). Mice were immunized with 16 μg of plasmid DNA lacking any insert (negative control), or with wild-type E7, N-CRT, N-CRT/E7, P-CRT/E7, C-CRT/E7 or CRT/E7 DNA on day 0 and were boosted with the same regimen on day 7. Matrigel® (Becton Dickinson and Co., Franklin Lakes, N.J.) was mixed with heparin (final concentration of 50 U/ml), bFGF (final concentration of 20 ng/ml), and VEGF (final concentration of 200 ng/ml) at 4° C. A total of 0.5 ml of this Matrigel® mixture was injected sc into the abdominal midline of DNA-vaccinated mice on day 7. Naive mice injected with Matrigel® mixed with heparin, bFGF and VEGF served as a positive control; naive mice injected with Matrigel® alone were negative controls. Mice were euthanized on day 16. The Matrigel® plugs were resected from surrounding connective tissues. Half of the plugs were fixed in 10% formaldehyde, embedded in paraffin, sectioned, and stained with hematoxylin and eosin or Giemsa stains to calculate MVD. In each section, the five most vascular areas were selected, microvessel were evaluated at 400× magnification, and the mean number in the five fields for the Matrigel® plugs was calculated (MVD count). The remaining half of each plug was assayed for hemoglobin content according to manufacturer's instructions (Drabkin's reagent kit; Sigma Diagnostics Co., St. Louis, Mo.).

Statistical Analysis

All results are expressed as means ±SE and are representative of at least two different experiments. Data for ICCS with flow cytometric analysis and tumor treatment experiments were analyzed by analysis of variance (ANOVA). Comparisons between individual data points were made using Student's t-test. Kaplan-Meier survival curves were used for tumor protection experiments for differences between curves, and p values were calculated using the log-rank test. p<0.05 was considered significant.

### Example 2

### Comparative Analysis of CRT/E7 DNA Vaccine with Other IPP's Linked to E7

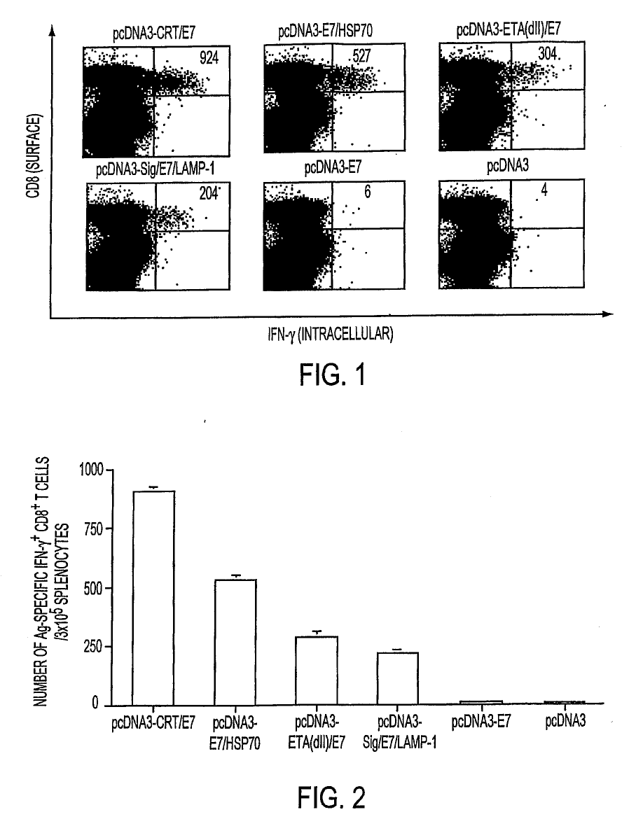

CD8+ T cell-mediated immune responses are important in controlling both HPV infections and HPV-associated neoplasms. To assess immune response to various DNA vectors, the frequency of E7-specific CD8⁺ T cell precursors generated by pcDNA3, pcDNA3-E7, pcDNA3-CRT/E7, pcDNA3-E7/HSP70, pcDNA3-ETA(dII)/E7, and pcDNA3-Sig/E7/LAMP-1 vaccine constructs, ICCS with flow cytometric analysis was done using spleen cells from vaccinated mice one week after the last vaccination. As shown in FIGS. 1 and 2, mice vaccinated with pcDNA3-CRT/E7 DNA exhibited the highest numbers of E7-specific IFN-γ⁺ CD8⁺ T cell precursors (per 3×10⁵ spleen cells)—655—compared to mice vaccinated with pcDNA3-E7/HSP70, pcDNA3-ETA(dII)/E7, pcDNA3-Sig/E7/LAMP-1, pcDNA3-E7, or pcDNA3 (p<0.05).

### Example 3

### Mice Immunized with CRT/E7 Vaccine Generate Potent Antitumor Responses

Therapeutic potential of the various chimeric DNA constructs were tested for treatment of an E7-expressing tumor, TC-1, using a previously described lung hematogenous spread model (Ji et al., supra). As shown in FIG. 3, mice given the pcDNA3-CRT/E7 vaccine exhibited significantly lower numbers of pulmonary nodules compared to mice vaccinated with pcDNA3 (negative control) or pcDNA3-E7 after TC-1 challenge (p<0.05). When comparing pcDNA3-CRT/E7 to pcDNA3-E7/HSP70, pcDNA3-ETA(dII)/E7, or pcDNA3-Sig/E7/LAMP-1 vaccines, pcDNA3-CRT/E7-immunized mice displayed lower mean numbers of pulmonary nodules than the others p<0.95).

### Example 4

### Vaccination with CRT/E7 Generates Higher Numbers of Antigen-Specific Memory T Cells and Provides Long-Term Tumor Protection

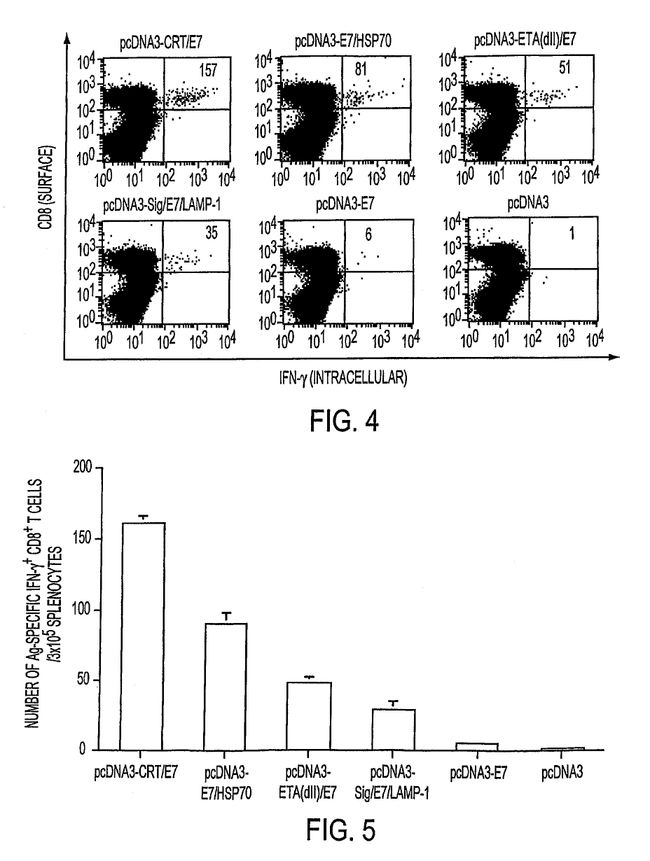

To measure E7-specific CD8⁺ memory T cell precursors generated by various DNA vaccines, animals were vaccinated with pcDNA3 (negative control), pcDNA3-E7 (antigen control), pcDNA3-CRT/E7, pcDNA3-E7/HSP70, pcDNA3-ETA(dII)/E7, and pcDNA3-Sig/E7/LAMP-1 DNA. ICCS flow cytometric were performed on spleen cells derived from vaccinated mice eight weeks after the initial vaccination. As shown in FIGS. 4 & 5, mice pcDNA3-CRT/E7 DNA induced the highest number of E7-specific IFN-γ⁺ CD8⁺ memory T cell precursors compared to mice vaccinated with the other DNA constructs (p<0.05).

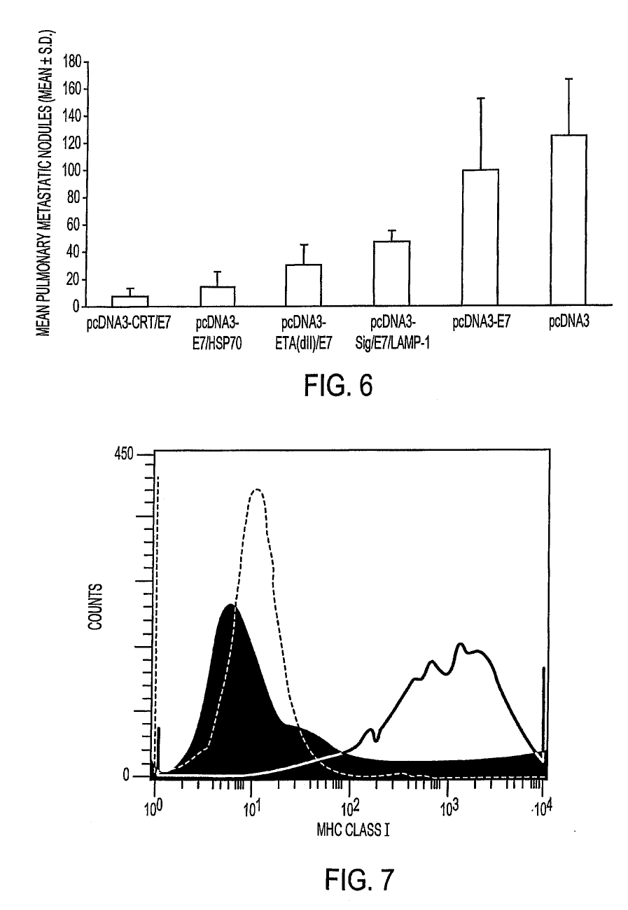

This vaccine also provided the best long-term protection against E7-expressing tumors. A long-term tumor protection study was done comparing even a larger number of IPP-Ag vaccine constructs: pcDNA3 (neg. control), pcDNA3-E7 (antigen control), pcDNA3-CRT/E7, pcDNA3-E7/HSP70, pcDNA3-VP22/E7 (Herpes simplex virus protein VP22), pcDNA3-ETA(dII)/E7, and pcDNA3-Sig/E7/LAMP-1 DNA. As shown in FIG. 6, mice vaccinated with the pcDNA3-CRT/E7 DNA exhibited significantly fewer pulmonary nodules compared to mice vaccinated with pcDNA3-Sig/E7/LAMP-1, pcDNA3-E7, or pcDNA3 (p<0.05). Although not statistically significant, the pcDNA3-CRT/E7 vaccinated mice also displayed lower mean numbers of pulmonary nodules than mice vaccinated with pcDNA3-E7/HSP70, pcDNA3-VP22/E7, pcDNA3-ETA(dII)/E7 (p<0.70).

### Example 5

### Vaccination with CRT/E7 DNA Results in Immunity that Controls E7-Expressing Tumors in which MHC Class I was Down-Regulated

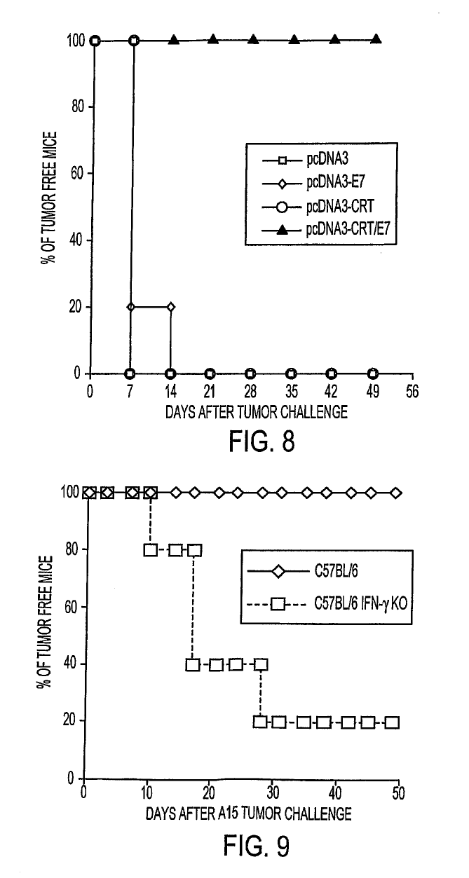

Since MHC class I expression is down-regulated in most cervical cancers, it was important to determine the effectiveness of the CRT/E7 DNA vaccines in protecting against tumors with low MHC class I expression. An E7-expressing murine tumor model with down-regulated MHC class I expression, termed TC-1 P3 (A15), was developed as described in Example 1. MHC class I expression in TC-1 P0 and TC-1 P3 (A15) tumor cells was done by flow cytometric analysis. As shown in FIG. 7, MHC class I expression was markedly down-regulated in TC-1 P3 (A15) compared to TC-1.

This was followed by a tumor protection study in C57BL/6 mice in which mice were vaccinated with 2 μg of CRT/E7 DNA, CRT DNA (CRT-only control), E7 DNA (antigen control), or vector alone (negative control), followed by challenge one week later with 5×10⁴ TC-1 P3 (A15) tumor cells. As shown in FIG. 8, all mice vaccinated with pcDNA3-CRT/E7 DNA were protected against tumor challenge with TC-1 P3 (A15) for a period of up to 45 days after tumor challenge. In contrast, all control mice (vaccinated with pcDNA3-CRT, pcDNA3-E7, or pcDNA3 vector) developed tumors within 14 days.

The present inventors and colleagues had previously shown that IFN-γ was essential for an anti-tumor effect generated by DNA vaccines employing Mycobacterium tuberculosis HSP70 linked to E7 (against an E7-expressing tumor cell line with down-regulated MHC class I (Cheng W F et al. Gene Ther 10: 1311-20, 2003. To determine whether if IFN-γ was also essential in the case of vaccination with CRT/E7 DNA, an experiment was done wherein wild type and IFN-γ KO mice were vaccinated and challenged with TC-1 P3 (A15). As shown in FIG. 9, whereas 100% of wild-type mice vaccinated with CRT/E7 were protected against challenge with TC-1 P3 (A15), only 20% of IFN-γ KO mice so vaccinated E7 were protected.

These foregoing results indicate that (a) immunization with CRT/E7 DNA can control E7-expressing tumors with down-regulated MHC class I expression and (b) that this protection is dependent upon the ability to make, express or upregulate IFN-γ (presumably in T cells).

### Example 6

### Intramuscular and Gene Gun Immunization with pNGVL4a-CRT/E7(detox) DNA Enhances E7-specific CD8+ T Cell Immune Responses

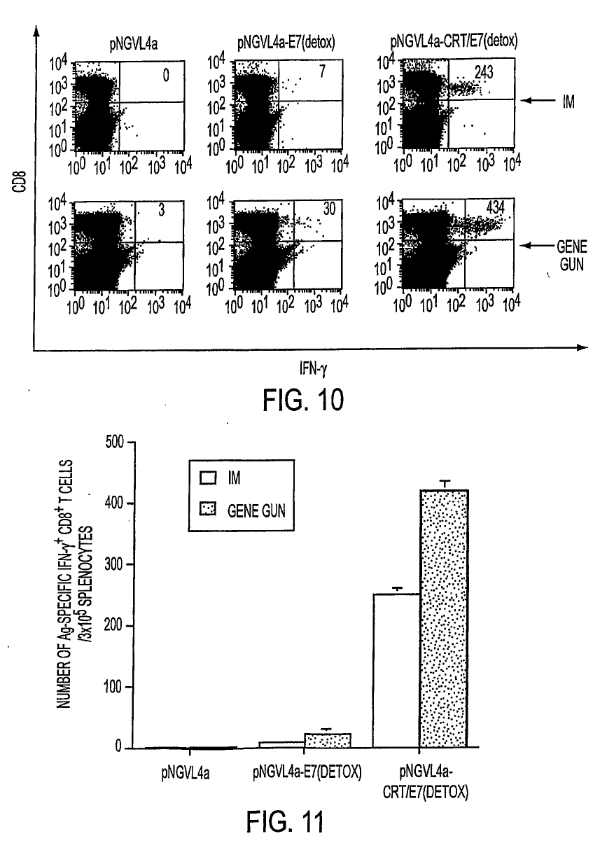

Because the pcDNA3-CRT/E7 DNA vector contains ampicillin resistance gene and the wild type E7 has a greater risk of oncogenically transforming host cells, a different vector more suitable for human use was employed. The present inventors used the pNGVL4a vector (see, for example, the present inventors' patent publication WO 2004/098526A2) to create a new CRT/E7 vector designated “pNGVL4a-CRT/E7(detox)” for use in the clinical setting. Moreover, since it is likely that human immunization will be performed by i.m. injection, a test was conducted to demonstrate that i.m. vaccination with pNGVL4a-CRT/E7(detox) in mice can enhance E7-specific CD8+ T cell immune responses. As shown in FIGS. 10 and 11, i.m. and gene gun immunization of mice with pNGVL4a-CRT/E7(detox) DNA significantly increased the number of E7-specific CD8+ T cell precursors compared to vaccination with an antigen control (pNGVL4a-E7(detox)) or negative vector control (pNGVL4a vector only) (p<0.05).

Gene gun-mediated immunization with pNGVL4a-CRT/E7(detox) generated more antigen-specific CD8+ T cell precursors than did i.m. immunization (p<0.05).

Furthermore, in the tumor protection experiments using E7-expressing tumors with down-regulated MHC class I expression (TC-1 P3 (A15)), the results indicated that mice vaccinated with pNGVL4a-CRT/E7(detox) DNA demonstrated 100% protection against TC-1 P3 (A15) up to 45 days after tumor challenge (not shown).

These results indicate that the pNGVL4a-CRT/E7(detox) DNA, like the pcDNA3-CRT/E7 DNA, is capable of markedly potentiating the number of E7-specific CD8+ T cells and resulting in a state in which E7-expressing tumors, even with down-regulated MHC class I expression, are controlled by the immune system. Thus, pNGVL4a-CRT/E7(detox) DNA is a useful vaccine construct for use in humans for control of HPV infections and HPV associated lesions.

### Example 7

### Discussion of Examples 1-6

E7-specific CD8+ T cell immune responses and anti-tumor effects were compared across generated by five effective chimeric DNA vaccines in which DNA encoded a fusion protein of the antigen and an IPP. The chimeric combinations tested were: CRT/E7, E7/HSP70, ETA(dII)/E7, Sig/E7/LAMP-1/E7) that the inventors' laboratory had previously developed

Mice vaccinated with pcDNA3-CRT/E7 construct generated the highest quantity of antigen-specific CD8+ T cell precursors and memory T cells, resulting in potent tumor therapeutic and long-term tumor protective effects.

The potentiated immune response induced by the CRT/E7 DNA vaccine could even control tumors with down-regulated MHC class I expression. This indicates that the vaccine would be useful for treating patients with cervical lesions in whom MHC class I expression was also down-regulated, a condition which normally facilitates evasion of a CD8+ CTL response by the tumor.

Also demonstrated herein is a pNGVL4a-CRT/E7(detox) DNA vaccine that has several advantages for clinical use over the pcDNA3 vaccine discussed above. The pNGVL4a-CRT/E7(detox) DNA vaccine, administrated via gene gun or intramuscularly, was able to potentiate the E7-specific CD8+ T cell immune response and control tumors with down-regulated class I.

Indeed, one concern about efficacy of immunotherapy is that tumors can evade immune responses through various mechanisms, including MHC class I down-regulation. A number of human cancers have been shown to downregulate MHC class I expression, including melanoma (Ferrone S et al., Immunol Today 16:487-94, 1995); lung cancer (Korkolopoulou P et al., Br J Cancer 73:148-53, 1996), prostate cancer (Sanda M G et al. J Natl Cancer Inst 87:280-5, 1995), breast cancer (Cabrera T et al. High frequency of altered HLA class I phenotypes in invasive breast carcinomas. Hum Immunol 50:127-34, 1996; Vitale M et al., Cancer Res; 58: 737-42, 1998), ovarian and colon cancer (Vegh Z et al., Cancer Res 53:2416-20, 1993) and cervical cancer (Connor M E et al., Int J Cancer 46:1029-34, 1990; Cromme F V et al. Oncogene 8:2969-75, 1993; Koopman L A et al. J Exp Med 191: 961-76, 2000).

By down-regulating MHC class I expression, tumor cells escape immune recognition; thus avoid killing by antigen-specific CD8⁺ T cells. The present invention provides a composition and method—using a CRT/E7 DNA vaccine against tumors with low MHC class I expression, —that generates a stronger immune response that is also able to overcome such evasion and thus be used for treating patients with advanced cervical cancer in which MHC class I expression is down-regulated.

Based on the present inventors previous studies with E7/HSP70, it was concluded that IFN-γ was required for tumor protection. IFN-γ could indeed up-regulate MHC class I expression on TC-1 P3 (A15) tumor cells to levels equivalent to “wild type” TC-1 P0 cells. The present discovery that CRT/E7 DNA vaccine required IFNγ for its immunopotentiating and anti-tumor action further supports a role for the stimulation of IFNγ expression (or an increase in the number of CD8+ T cells expressing cytoplasmic IFNγ in an anti-tumor immune response against tumor cells in which MHC class I expression was down-regulated.

Tests of a novel immunogenic vector, pNGVL4a-CRT/E7(detox), showed that successful immunization could be achieved by both the i.m. route and by i.d. gene gun administration, leading to both an increase in the numbers of CD8⁺ T cells and efficacy against a tumor in which MHC class I is down-regulated. Intradermal immunization via gene gun directly targets antigen to professional APCs, Langerhans cells, allowing the intracellular strategy to further improve direct presentation of antigen to T cells by DNA-transfected DCs. In comparison, intramuscular immunization likely targets antigen to myocytes, and the antigen encoded by DNA vaccine is eventually presented through bone marrow-derived APCs through the cross-priming mechanism. Vaccination with pNGVL4a-CRT/E7(detox) likely led to secretion of chimeric CRT/E7(detox) protein or lysis of cell expressing CRT/E7(detox) antigen, releasing the chimeric protein from cells to be taken up and processed by nearby APCs via the MHC class I-restricted pathway. The linkage of CRT to E7 facilitates cross-priming of E7 antigen. One recent study found that CD91, an α2 macroglobulin receptor commonly expressed on professional APCs, serves as a receptor for HSPs and CRT, and facilitates cross-priming (Basu S et al., Immunity 14:303-13, 2001). Intramuscular immunization of DNA vaccines encoding CRT/E7(detox) may promote prolonged release of the CRT/E7(detox) protein from transfected cells to target, concentrate CRT/E7(detox) to professional APCs, and facilitate the cross-priming of an E7-specific response. Therefore different routes of administration may generate different degrees of immune responsivity by the same vaccine.

In summary, CRT/E7 DNA vaccines are attractive therapeutic vaccines not only because of the ability to generate effective anti-tumor immunity against E7-expressing tumors and to control E7-expressing tumors with down-regulated MHC class I expression in vaccinated mice, but also because of the ability to prevent or delay tumor growth by targeting tumor vasculature.

It should be evident that the utility of a CRT/E7 DNA vaccine (particularly using E7(detox) is not limited to treating patients with E7-expressing cervical cancers but is also useful to treat subjects who are in need of anti-angiogenic therapy that is mediated by CRT independent of the antigen with which it is linked. The same is true of a related vaccine employing CRT/E6, or any other antigen for that matter.

### Example 8

### DNA Vaccines Encoding CRT Domains Elicit Potent T Cell-Mediate Immune Responses Anti-Tumor Immunity and Inhibition of Angiogenesis

(This Example specifically incorporates by reference Cheng et al., Vaccine 23 3864-3874, 2005)

Vaccination with DNA encoding N-CRT, P-CRT, or C-CRT linked to E7 significantly enhances the E7-specific CD8+ T cell response. To determine if the different domains of calreticulin when linked with the E7 DNA vaccines could enhance E7-specific T cell-mediated immune responses in mice, we performed ICCS with flow cytometric analysis to characterize E7-specific CD8⁺ and CD4⁺ T cell precursors generated as a result of immunization with CRT/E7 DNA or DNA encoding CRT domains linked to E7. Vaccination with each of N-CRT/E7, P-CRT/E7, and C-CRT/E7 DNA generated higher frequencies of E7-specific IFN-γ-secreting CD8⁺ T cell precursors when compared to vaccination with E7 DNA (p<0.01). Physical linkage of N-CRT to E7 was required for this enhancement since administration of a mixture of DNA encoding the N-CRT and E7 DNA did not enhance CD8⁺ T cell activity. Vaccination with CRT/E7 DNA generated a slightly higher number of E7-specific CD8⁺ T cell precursors (220.5±18.5) when compared to the N-CRT/E7 (178.0±18.5), P-CRT/E7 (140.0±16.0) and C-CRT/E7 (128.0±10.0) (p<0.01). Thus DNA encoding each CRT domain has immunopotentiating activity, and can serve as an IPP when linked to DNA encoding antigen. However, none of CRT domains linked to E7 stimulated more E7-specific IFN-γ-secreting CD4⁺ T cells (compared to vaccination with E7 DNA).

Vaccination with N-CRT/E7 DNA significantly enhanced the E7-specific antibody response. Vaccination with CRT/E7 or N-CRT/E7, but not with P-CRT/E7 nor C-CRT/E7 DNA enhanced E7-specific antibody responses in vaccinated mice compared to vaccination with wild type E7 DNA (p<0.01). There was no significant difference between the titers of anti-E7 antibodies induced by N-CRT/E7 and vs. CRT/E7

Vaccination with N-CRT/E7, P-CRT/E7 or C-CRT/E7 DNA enhanced E7-specific Tumor Protection. In vivo tumor protection experiment were carried out as above using TC-1 tumor cells. 100% of mice receiving N-CRT/E7, P-CRT/E7, C-CRT/E7, or CRT/E7 DNA remained tumor-free 60 days after TC-1 challenge. In comparison, all mice vaccinated with wild-type E7 DNA only developed tumors within 14 days of challenge. Thus, each domain of CRT can protect vaccinated mice against a lethal challenge with E7-expressing tumor cells when linked to the E7 antigen in a DNA vaccine.

Treatment with N-CRT/E7, P-CRT/E7, or C-CRT/E7 DNA Significantly Reduced Pulmonary Tumor Nodules in C57BL/6 Mice. The therapeutic potential of the above vaccines was tested using a the lung hematogenous spread model described above. C57BL/6 mice treated with N-CRT/E7 DNA (1.0±0.4), P-CRT/E7 (1.2±0.8), or C-CRT/E7 (1.4±0.6) all exhibited significantly fewer pulmonary tumor nodules than did mice treated with “control” DNA vaccines (a) wild-type E7 (antigen only) (139.0±11.0) or (b) “no antigen” N-CRT (34.0±3.2) by one-way ANOVA (p<0.001). Thus DNA encoding each of the 3 CRT domains when linked with E7 DNA generated potentiated antitumor effects in a lung hematogenous spread model.

Treatment of mice with N-CRT DNA also resulted in significantly fewer tumor nodules than treatment with wild-type E7 DNA or no treatment (one-way ANOVA, p<0.001), suggesting that N-CRT is capable of inducing non-immunological antitumor effects.

Treatment with N-CRT/E7 DNA or N-CRT DNA Significant Reduced Pulmonary Tumor Nodules in Immunocompromised Mice. To study this apparently T cell-independent (likely non-immunological) antitumor effect, immunocompromised BALB/c athymic nude mice (nu/nu) given N-CRT, N-CRT/E7, or CRT/E7 DNA displayed a significantly lower mean number of pulmonary tumor nodules (18.0±2.0 for N-CRT, 25.0±4.0 for N-CRT/E7) compared with mice treated with wild type E7 DNA (215.0±10.0), plasmid without insert (217.5±17.0), or untreated naïve mice (230.0±22.5) (one-way ANOVA, p<0.001). Interestingly, neither P-CRT/E7 nor C-CRT/E7 stimulated significant reduction of tumor nodules compared to mice treated with wild-type E7 DNA. In addition, nude mice treated with N-CRT/E7 DNA exhibited significantly fewer pulmonary tumor nodules than nude mice treated with CRT/E7 DNA (one-way ANOVA, p<0.05). Thus, treatment with N-CRT, N-CRT/E7, or CRT/E7 DNA can generate an antitumor effects even in the absence of T cell-mediated immune responses, whereas P-CRT/E7 and C-CRT/E7 DNA did not have this effect.

CRT/E7, N-CRT/E7 and N-CRT Reduced Microvessel Density (MVD) in Tumors of BALB/c nude Mice. To evaluated the possibility that inhibition of angiogenesis could explain the above non-immunologic anti-tumor effects, MVD was evaluated in the pulmonary tumors of nude mice treated with various DNA vaccines. Treatment of nude mice with N-CRT, N-CRT/E7, or CRT/E7 DNA significantly reduced MVD in pulmonary tumors than did treatment with wild-type E7, P-CRT/E7 or C-CRT/E7 group (one-way ANOVA, p<0.001). N-CRT/E7 DNA treatment was more potent than CRT/E7 DNA (one-way ANOVA, p<0.05). Taken together, the results demonstrate that T cell-independent antitumor effects elicited by vaccination with N-CRT, N-CRT/E7, or CRT/E7 DNA occurs via inhibition of tumor angiogenesis, an action that was associated in particular with the N-domain of CRT.

Reduced MVD and Hemoglobin (Hb) in Matrigel® Plugs from Mice Challenged with TC-1 tumor Cells and treated with N-CRT, N-CRT/E7, or CRT/E7 DNA. A more quantitative assessment of antiangiogenesis in mice treated with the above DNA constructs, were performed in a Matrigel®-based in vivo angiogenesis assays. The Hb content of Matrigel implants from N-CRT, N-CRT/E7, or CRT/E7-treated mice were significantly lower than those from mice treated with insertless control DNA, E7 DNA, P-CRT/E7 DNA, or C-CRT/E7 DNA (p<0.01, ANOVA). This assay revealed that N-CRT or N-CRT/E7 DNA inhibited bFGF- or VEGF-induced in vivo angiogenesis. The Hb levels Matrigel® implants from N-CRT/E7-treated mice were significantly lower than those from CRT/E7-treated mice (p<0.01, ANOVA).

The MVD in these Matrigel® samples serve as an additional measure of angiogenesis inhibition, since angiogenesis permits red blood cells (source of Hb) to extravasate from vessels. The mean MVDs in Matrigel® samples from N-CRT (23.7±10.4), N-CRT/E7 (21.3±4.7), and CRT/E7 (29.0±9.2) DNA-treated mice were similar to one another, but significantly lower than the samples from mice treated with control plasmid DNA (no insert) (98.3±31.8), wild-type E7 DNA (76.7±12.0), C-CRT/E7 DNA (77.3±9.6), or P-CRT/E7 DNA (76.3±6.7). This confirms that the N domain of CRT is responsible for the observed anti-angiogenic effects.

N-CRT DNA Must be Linked to E7 DNA for Antitumor Effects. To assess whether the linkage of N-CRT to E7 is essential for the antitumor effect against E7-expressing tumors in vaccinated mice, we performed in vivo tumor protection experiments using TC-1 tumor cells. Mice were vaccinated with mixtures of DNA constructs that encoded E7 DNA and separately, constructs encoding, N-CRT DNA, N-CRT/E7 or N-CRT DNA. At the appropriate time, mice were challenged with E7-bearing TC-1 tumor cells. All mice vaccinated with the N-CRT/E7 DNA vaccine remained tumor-free. In contrast, all mice vaccinated with the other DNA vaccine combinations (including a mixture of N-CRT DNA and E7 DNA) developed tumors within 2 weeks of challenge. This proves that linkage of N-CRT to E7 was required for the observed antitumor effects in vaccinated mice.

CD8+ T cells, but not CD4+ T cells or NK cells, are Important for the Antitumor effect Evoked by N-CRT/E7 DNA vaccine. In vivo antibody depletion experiments were conducted delete individual T cell subsets or NK cells. N-CRT/E7 DNA-vaccinated mice that had been depleted of CD8+ T cells, as well as all unvaccinated naive mice, grew tumors within 14 days. In contrast, all N-CRT/E7 DNA-vaccinated mice depleted of CD4+ T cells or of NK cells remained tumor free 56 days after challenge. These results showed that suggest that in response to N-CRT/E7 DNA vaccine CD8+ T cell s, but not CD4+ T cells or NK cells, mediated the antitumor immunity.

Discussion

Thus, the present inventors demonstrated that linkage of DNA encoding N-CRT, P-CRT, or C-CRT to DNA encoding the HPV-16 E7 antigen can significantly enhance the potency of an E7-expressing DNA vaccine. Each domain of CRT linked with E7 DNA elicited strong E7-specific CD8⁺ T cell immune responses, generated significant CD8⁺ T cell-dependent protective effects against subcutaneous HPV-16 E7-expressing tumors, and could effectively treat lethal pulmonary tumor nodules. In comparison, vaccination with either N-CRT/E7 or CRT/E7 DNA significantly enhanced E7-specific antibody responses. Only DNA vaccines encoding the N domain inhibited angiogenesis leading to therapeutic independent of the immune response. Thus, a DNA vaccine encoding N-CRT (including full length CRT) linked to a tumor antigen represents a useful approach for treatment that combines immunotherapeutic and antiangiogenic approaches for increasing antitumor effects.

The mechanism for CRT (or CRT domain) immunological effects may include enhancing MHC class I processing of antigen by targeting linked antigen to the ER in transfected APCs such as DCs. Another possible mechanism for these effects is “cross-priming”, whereby secretion of chimeric protein or lysis of cells expressing chimeric antigen releases the chimeric protein exogenously to be taken up and processed by other APCs via the MHC class I restricted pathways. CD91, an α2 macroglobulin receptor, serves as a receptor for heat shock proteins, including CRT, gp96, HSP70 and HSP90 and facilitate the cross-priming effects. There are still questions about which of the three CRT domains binds CD91. Both direct and cross-priming mechanisms may also explain the immunopotentiating effects or P-CRT/E7 DNA and C-CRT/E7 DNA.

Even though antibody-mediated responses are not considered to play an important role in controlling HPV-associated malignancies, antigen-specific Abs are significant in other tumor disease, such as the treatment of HER-2/neu antigen-bearing breast cancer cells. The chimeric N-CRT or CRT vaccine strategy described for E7 may be directly transferred to Her-2/neu epitopes to stimulate HER-2/neu-specific antibody responses that can arrest growth of cells expressing high levels of surface HER-2/neu (Harwerth, I M et al., Br J Canc 68:1140-5, 1993).

By inhibiting angiogenesis, including endothelial cell growth, N-CRT DNA, N-CRT/E7 DNA or N-CRT linked to any other antigen may be employed to reduce tumor neovascularization and inhibit tumor growth. N-CRT/E7 and CRT/E7 are two chimeric molecules that can control established tumors cells through E7-specific CD8+ T cell-mediated immune responses and inhibit the growth of tumor vasculature through antiangiogenesis. Effective antiangiogenic effects require repeated and high dose administration of DNA encoding CRT or N-CRT. A single DNA vaccination results in peak serum CRT levels at 7 days post vaccination, which then taper to near-baseline levels within 14 days post vaccination (Xiao, F., et al. Gene Ther 9:1207-13, 2002,). The level of serum CRT depends on the dose of CRT DNA given. Typical DNA vaccine doses (2 μg) did not result in detectable serum CRT levels. Repeated, high-dose CRT DNA vaccination does lead to detectable levels of serum CRT and anti-angiogenic effects in vaccinated mice (Cheng et al., 2001, m supra). Undesired side effects of angiogenesis inhibition include diminution of wound healing. However, this does not appear to occur at tumor-inhibiting doses (Lange-Asschenfeldt, B et al., J Invest Dermatol 117:1036-41, 2001). Furthermore, additional experiments by the present inventors showed that vaccination with the CRT/E7 DNA vaccine did not inhibit wound healing or cause any pathologic changes in the major organs of mice.

All references cited above are all incorporated by reference herein, in their entirety, whether specifically incorporated or not. All publications, patents, patent applications, GenBank sequences and ATCC deposits, cited herein are hereby expressly incorporated by reference for all purposes.

Having now fully described this invention, it will be appreciated by those skilled in the art that the same can be performed within a wide range of equivalent parameters, concentrations, and conditions without departing from the spirit and scope of the invention and without undue experimentation.

1. A nucleic acid molecule that is an expression vector expressable in a eukaryotic cell, and encodes a chimeric or fusion polypeptide, which molecule comprises:
(a) a first nucleic acid sequence encoding a first polypeptide which is calreticulin (CRT) or a biologically active homologue, domain or fragment thereof,
which homologue, domain or fragment (i) forms complexes with peptides in vitro; (ii) when expressed in a cell, participates in folding and assembly of nascent glycoproteins, (iii) when expressed in a cell, associates with peptides transported into the endoplasmic reticulum by transporters that are associated with antigen processing, and/or (iv) inhibits angiogenesis;
(b) a second nucleic acid sequence that is linked in frame to said first nucleic acid sequence and that encodes an antigenic polypeptide or peptide; and
(c) operably linked thereto, a promoter active in said eukaryotic cell and, optionally, one or more regulatory elements that enhance expression of said nucleic acid in said cell.

2. The nucleic acid molecule of claim 1, wherein the antigenic peptide comprises an epitope that binds to a MHC class I protein.

3. The nucleic acid molecule of claim 1, wherein the first polypeptide is encoded by SEQ ID NO:10 or a fragment thereof that encodes a biologically active domain or fragment of said polypeptide.

4. The nucleic acid molecule of claim 3, wherein the first nucleic acid sequence encodes one or more CRT fragments or domain selected from the group consisting of (a) N-CRT, (b) P-CRT, (c) S-CRT and (d) a biologically active variant of (a), (b) or (c) but does not encode full length CRT.

5. The nucleic acid molecule of claim 4, wherein the first nucleic acid sequence encodes N-CRT (SEQ ID NO:14), P-CRT (SEQ ID NO:15), S-CRT (SEQ ID NO:16) or a homologue of N-CRT, P-CRT or S-CRT.

6. The nucleic acid molecule of claim 4, wherein the first nucleic acid sequence encodes N-CRT (SEQ ID NO:14).

7. The nucleic acid molecule of claim 4, wherein the first nucleic acid sequence encodes any two or more of N-CRT (SEQ ID NO:14), P-CRT (SEQ ID NO:15), C-CRT (SEQ ID NO:16) or any combination thereof.

8. The nucleic acid molecule of claim 1 wherein the antigen is one which is present on, or cross-reactive with an epitope of, a pathogenic organism, cell, or virus.

9. The nucleic acid molecule of claim 8, wherein the virus is a human papilloma virus.

10. The nucleic acid molecule of claim 9, wherein the antigen is an E7 or E6 polypeptide of HPV-16 having the sequence SEQ ID NO:3 or SEQ ID NO:6, respectively, an -in-frame linked combination of E6 and E7, an antigenic fragment of E6 or E7, or non-oncogenic mutant or variant of E6 or E7; or an in-frame linked combination of a non-oncogenic mutant or variant of E6 and of E7.

11. The nucleic acid molecule of claim 10, wherein the HPV-16 E7 polypeptide is a non-oncogenic mutant or variant of said E7 polypeptide.

12. The nucleic acid molecule of claim 9 wherein the sequence of the non-oncogenic mutant or variant E7 polypeptide differs from SEQ ID NO:3 by one or more of the following substitutions:
(a) Cys at position 24 to Gly or Ala;
(b) Glu at position 26 to Gly or Ala; and
(c) Cys at position 91 to Gly or Ala.

13. The nucleic acid molecule of claim of claim 12 wherein the sequence of the non-oncogenic mutant or variant E7 polypeptide is sequence SEQ ID NO:4.

14. The nucleic acid molecule of claim 10, wherein the antigen is the E6 polypeptide having the sequence SEQ ID NO:6 or an antigenic fragment thereof.

15. The nucleic acid molecule of claim 11, wherein the HPV-16 E6 polypeptide is a non-oncogenic mutant or variant of said E6 polypeptide.

16. The nucleic acid molecule of claim 15 wherein the sequence of the non oncogenic mutant or variant E6 polypeptide differs from SEQ ID NO:6 by one or more of the following substitutions:
(a) Cys at position 63 to Gly or Ala;
(b) Cys at position 106 to Gly or Ala; and
(c) Ile at position 128 to Thr.

17. The non oncogenic mutant E6 polypeptide of claim 15 having the sequence SEQ ID NO:7.

18. The nucleic acid molecule of claim 10 wherein the antigen is a linked, in-frame combination of E7 and E6 polypeptide, an antigenic fragment thereof, or a non-oncogenic mutant or variant of E7 and E6.

19. The nucleic acid molecule of any of claims 1 that is part of a plasmid.

20. The nucleic acid molecule of claim 19 wherein said plasmid is pNGV4a.

21. The nucleic acid molecule of claim 1 that is characterized as pNGVL4a/CRT/E7(detox), and has the sequence SEQ ID NO:20.

22. A pharmaceutical composition capable of inducing or enhancing an antigen-specific immune response, comprising:
(a) pharmaceutically and immunologically acceptable excipient in combination with;
(b) a composition comprising the nucleic acid molecule of claim 1.

23-25. (canceled)

26. A method of inducing or enhancing an antigen specific immune response in a subject comprising administering to the subject an effective amount of the pharmaceutical composition of claim 22, thereby inducing or enhancing said response.

27-35. (canceled)

36. The method of claim 26 wherein said administering is by a intramuscular injection by gene gun administration or by needle-free jet injection.

37-40. (canceled)

41. A method of inhibiting growth or preventing re-growth of a tumor expressing HPV E7 or E6 protein in a subject, comprising administering to said subject an effective amount of a pharmaceutical composition of claim 22, wherein said second nucleic acid sequence encodes one or more epitopes of E7 or E6, respectively, thereby inhibiting said growth or preventing said re-growth.

42-53. (canceled)
